(19) United States
(12) Patent Application Publication
(10) Pub. No.: US 2013/0302882 A1
(43) Pub. Date: Nov. 14, 2013

(54) METHOD FOR STERILIZING SEPARATION MEMBRANE MODULES, STERILIZATION DEVICE, AND APPARATUS FOR PRODUCING CHEMICALS

(71) Applicants: Makoto Nishida, Otsu (JP); Masahiro Henmi, Otsu (JP); Norihiro Takeuchi, Otsu (JP); Satoko Kanamori, Otsu (JP); Jihoon Cheon, Otsu (JP)

(72) Inventors: Makoto Nishida, Otsu (JP); Masahiro Henmi, Otsu (JP); Norihiro Takeuchi, Otsu (JP); Satoko Kanamori, Otsu (JP); Jihoon Cheon, Otsu (JP)

(73) Assignee: TORAY INDUSTRIES, INC, Tokyo (JP)

(21) Appl. No.: 13/997,094

(22) PCT Filed: Dec. 22, 2011
(86) PCT No.: PCT/JP2011/079827

§ 371 Date: Jun. 21, 2013

(30) Foreign Application Priority Data
Dec. 24, 2010 (JP) 2010-287133

(51) Int. Cl. A61L 2/04

(52) U.S. Cl. CPC A61L 2/04
USPC 435/286.5; 422/38; 422/109

(57) ABSTRACT

A method of sterilizing a separation membrane module reduces damage of a ceramic separation membrane, a sterilization process device achieves the method of sterilizing, and an apparatus produces chemicals. The method of sterilizing the separation membrane module includes a ceramic-containing separation membrane including a temperature raise step of raising the temperature of the separation membrane module to a predetermined sterilization temperature by feeding sterilization water having a controlled temperature and pressure so that a rate of temperature change of the separation membrane module is 6.0° C. or less per minute, and a sterilization step of sterilizing the separation membrane module at a predetermined temperature for a predetermined time.

### TECHNICAL FIELD

This disclosure relates to a method of sterilizing a separation membrane module including at least a ceramic-containing separation membrane, a sterilization device that carries out the method of sterilizing the separation membrane module, and an apparatus for producing chemicals.

### BACKGROUND

The ceramic-containing separation membrane (hereinafter, abbreviated as a “ceramic membrane”) has excellent physical strength and chemical strength and can precisely control the pore diameter compared with an organic polymer membrane. Form these viewpoints, the ceramic membrane can be preferably used for the purpose of solid-liquid separation and the like. Particularly, the ceramic membrane is difficult to deteriorate after cleaning with strong acid or strong alkali, and thus, can be preferably used for a process of liquid containing a lot of suspending solids, in which the separation membrane is frequently cleaned. As an example of raw liquid containing a lot of suspending solids, solutions containing food products such as dairy products, solutions of pharmaceutical products or the likes can be named.

Japanese Examined Patent Application No. 7-12303 develops a technique for continuous fermentation of lactic acid bacteria using a ceramic membrane as a separation membrane. In production of chemicals by the continuous fermentation, the culture is required to be carried out in a state that required sections in the device are sterilized and contamination is prevented. However, Japanese Examined Patent Application No. 7-12303 only describes that heating sterilization is possible because the separation membrane is the ceramic membrane. Specific means of the heating sterilization is not described in Japanese Examined Patent Application No. 7-12303.

As methods of sterilizing; flame sterilization, steam sterilization, hot-water sterilization, ultraviolet ray sterilization, gamma ray sterilization, and gas sterilization can be exemplified. Among these methods, the flame sterilization, the ultraviolet ray sterilization, and the gamma ray sterilization are not adequate for the method for sterilizing the separation membrane module because uniform sterilization of the ceramic membrane is difficult. In the gas sterilization, which introduces ethylene oxide gas or other gas into the separation membrane module, the gas may remain in the separation membrane module and also may affect properties of filtrate, and thus the gas sterilization is not adequate for the method of sterilizing a separation membrane module. For these reasons, the steam sterilization and the hot-water sterilization are preferably used for the method of sterilizing the ceramic membrane.

Japanese Patent Application Laid-open No. 6-38728 introduces a technique of carrying out continuous fermentation of fermented liquor using a ceramic membrane. To prevent contamination into the fermented liquor, Japanese Patent Application Laid-open No. 6-38728 describes that the ceramic membrane is used after steam sterilization. However, whereas the ceramic membrane has high upper temperature limit, it has a drawback of weakness to rapid temperature change. There have been problems that rapid contact with high temperature vapor used for sterilization causes cracks of the ceramic membrane and deterioration in cutoff property that the ceramic membrane has.

It could therefore be helpful to provide a method of sterilizing a separation membrane module in which a sterilization process can be carried out while preventing damage of the separation membrane module including at least a ceramic-containing separation membrane, a sterilization device that carries out the method of sterilizing a separation membrane module, and an apparatus for producing chemicals.

### SUMMARY

We thus provide a method of sterilizing a separation membrane module, including a separation membrane containing at least ceramic, by using sterilization water, which includes: a temperature raising step of raising a temperature of the separation membrane module to a predetermined sterilization temperature by feeding the sterilization water to the separation membrane module and controlling temperature and pressure of the fed sterilization water so that the temperature of the separation membrane module rises at 6.0° C. or less per minute; and a sterilization step of sterilizing the separation membrane module at a predetermined temperature for a predetermined time after the temperature of the separation membrane module reaches the predetermined sterilization temperature.

Moreover, the method further includes: a temperature measurement step of measuring a temperature T of the separation membrane module; and an initial temperature control step of controlling T and/or a temperature Tw of the sterilization water so that Tw satisfies |T−Tw|≦30.0° C., wherein the temperature raising step is carried out after the initial temperature control step.

Moreover, the temperature measurement step measures a temperature of an upstream of the separation membrane module which is a fed side of raw liquid being a process target as the temperature T; and the temperature raising step and the sterilization step feed the sterilization water to the upstream of the separation membrane module.

Moreover, the temperature measurement step measures any one of temperatures T of an upstream and a downstream of the separation membrane module as the temperature T; and the temperature raising step and the sterilization step feed the sterilization water to the upstream and downstream of the separation membrane module.

Moreover, a method of sterilizing a separation membrane module includes a separation membrane containing at least ceramic, by using sterilization water as set forth above, includes: a temperature raising step of raising a temperature of the separation membrane module to a predetermined sterilization temperature by feeding the sterilization water to an upstream where raw liquid being a process target of the separation membrane module is fed and to a downstream where filtrate after processing is collected and controlling a temperature and a pressure of the fed sterilization water so that the temperature of the upstream and the downstream of the separation membrane module rises at 6.0° C. or less per minute; and a sterilization step of sterilizing the separation membrane module at a predetermined temperature for a predetermined time after the temperature of the upstream and the downstream of the separation membrane module reaches the predetermined sterilization temperature.

Moreover, the method further includes: a temperature measurement step of measuring a temperature T1 of the upstream and a temperature T2 of downstream of the separation membrane module; and an initial temperature control step of controlling the T1 and/or the T2 and/or a temperature Tw1 of the sterilization water fed to the upstream of the separation membrane module and temperature Tw2 of the sterilization water fed to the downstream of the separation membrane module so that the Tw1 and Tw2 satisfy |T1−Tw1|≦30.0° C. and |T2−Tw2|≦30.0° C., wherein the temperature raising step is carried out after the initial temperature control step.

Moreover, the method further includes a cooling step of cooling the separation membrane module after the sterilization step so that temperature of the separation membrane module drops at 6.0° C. or less per minute.

Moreover, a sterilization device that sterilizes a separation membrane module comprising a separation membrane comprising at least ceramic, includes: a temperature measurement means to measure a temperature of the separation membrane module; and a sterilization water control part to generate liquid phase or gas phase sterilization water having a controlled temperature and pressure and to feed the sterilization water to the separation membrane module, wherein the sterilization water control part feeds the sterilization water so that temperature of the separation membrane module rises or drops at 6.0° C. or less per minute.

Moreover, an apparatus for producing chemicals includes: a separation membrane module comprising a separation membrane comprising at least ceramic; the sterilization device as described above; a fermentation tank that converts a feedstock into fermented liquid containing chemicals by fermentation culture of the feedstock by microorganisms; and a fermented liquid circulation means that sends the fermented liquid from the fermentation tank to the separation membrane module.

By carrying out the method of sterilizing a separation membrane module described above, temperature difference between the separation membrane module and sterilization water at the time of start of the sterilization can be reduced in the separation membrane module including the ceramic membrane, and thus, moderate temperature change in the separation membrane module is maintained. This enables reduction of damage of the ceramic membrane associated with rapid temperature change

### BRIEF DESCRIPTION OF THE DRAWINGS

FIG. 1 is a schematic view of a sterilization device of a separation membrane module according to a first example.

FIG. 2 is a view illustrating an aspect of the separation membrane module used in the first example.

FIG. 3A is a cross-sectional view and FIG. 3B is a side view of a monolith membrane in the separation membrane module of FIG. 2.

FIG. 4 is a flowchart of a sterilization process according to the first example.

FIG. 5 is a schematic view of a sterilization device of the separation membrane module according to a first modification of the first example.

FIG. 6 is a flowchart of a sterilization process according to a second modification of the first example.

FIG. 7 is a schematic view of a sterilization device of a separation membrane module according to a second example.

FIG. 8 is a flowchart of a sterilization process according to the second example.

FIG. 9 is a flowchart of a sterilization process according to a first modification of the second example.

FIG. 10 is a schematic view of a sterilization device of a separation membrane module according to a second modification of the second example.

FIG. 11 is a schematic view of a sterilization device of a separation membrane module according to a third modification of the second example.

FIG. 12 is a schematic view of an apparatus for producing chemicals according to a third example.

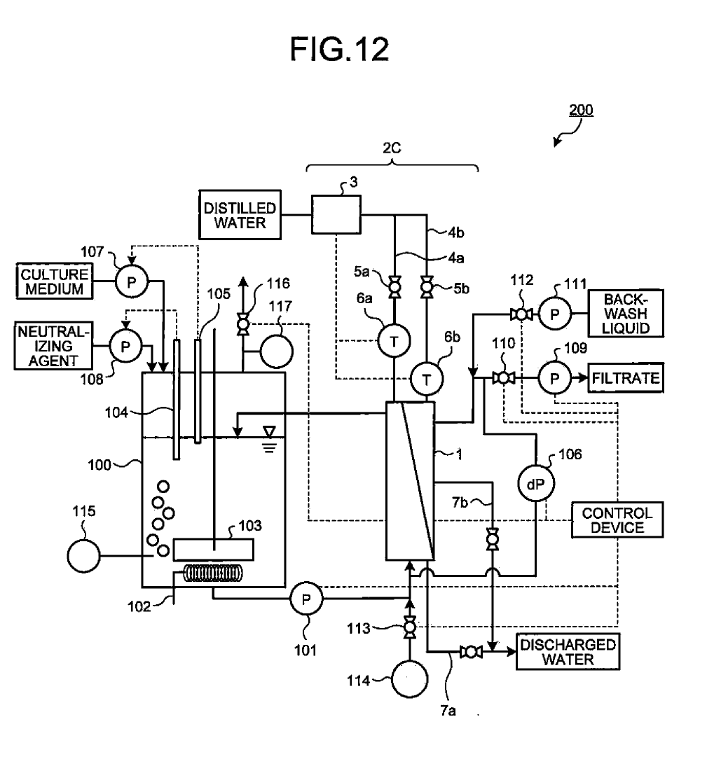

### REFERENCE SIGNS LIST

1 Separation Membrane Module
2, 2A, 2B, 2C, and 2D Sterilization Device
3 Sterilization Water Control Part
   3a Sterilization Water Control Part (Upstream)
   3b Sterilization Water Control Part (Downstream)
4 Sterilization Water Feed Line
   4a Sterilization Water Feed Line (Upstream)
   4b Sterilization Water Feed Line (Downstream)
5 Temperature Measurement Part
   5a Temperature Measurement Part (Upstream)
   5b Temperature Measurement Part (Downstream)
6 Valve
   6a Valve (Upstream)
   6b Valve (Downstream)
7 Sterilization Water Discharge Line
   7a Sterilization Water Discharge Line (Upstream)
   7b Sterilization Water Discharge Line (Downstream)
8 Sterilization Water Feed Pump
10 Ceramic Membrane
11 Module Container
12 O-ring
13 Liquid Flow Opening
   13a Raw Liquid Feed Opening/Concentrated Liquid Discharge Opening
   13b Filtrate Discharge Opening/Backwash Liquid Feed Opening
20 Ceramic Substrate
21 Through-bore
22 Separation Function Layer
23 Corrective Slit
24 Corrective Bore
25 Corrective Slit Communication Bore
100 Fermentation Tank
101 Circulation Pump
102 Temperature Control Device
103 Stirring Device
104 pH Sensor/Control Device
105 Level Sensor/Control Device
106 Pressure Difference Sensor
107 Culture Medium Feed Pump
108 Neutralizing Agent Feed Pump
109 Filtration Pump
110 Filtration Valve
111 Backwash Pump
112 Backwash Valve
113 Piping Gas Feed Control Valve
114 Piping Scrubbing Gas Feed Device
115 Fermentation Tank Gas Feed Device
116 Fermentation Tank Pressure Adjustment Valve
117 Fermentation Tank Pressure Gauge
200 Apparatus for Producing Chemicals

### DETAILED DESCRIPTION

### First Example

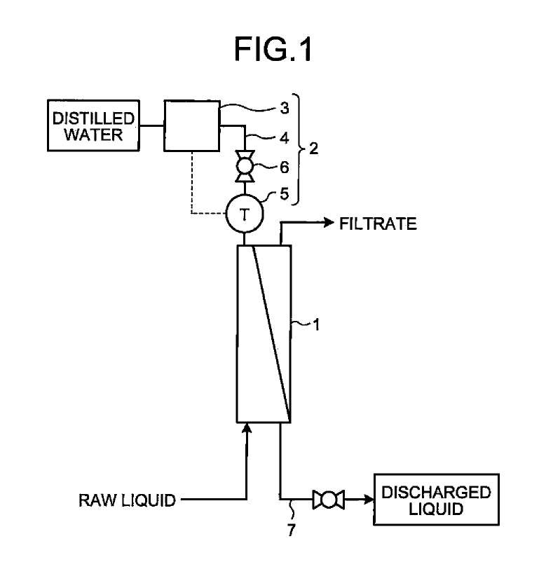

A method of sterilizing a separation membrane module and a sterilization device will be described with reference to FIG. 1. FIG. 1 is a schematic view for exemplifying the sterilization device for the separation membrane module according to a first example. In this sterilization device 2, a separation membrane module 1 is sterilized by flowing sterilization water to an upstream of the separation membrane module 1 including a ceramic membrane. The sterilization device 2 includes a sterilization water control part 3, a sterilization water feed line 4, a temperature measurement part 5, and a valve 6. The sterilization water control part 3 and the upstream of the separation membrane module 1 are communicated through the sterilization water feed line 4. The temperature measurement part 5 is located at a position where temperature in the upstream of the separation membrane module 1 can be measured. The valve 6 is located between the sterilization water control part 3 and the temperature measurement part 5. Temperature information obtained in the temperature measurement part 5 is sent to the sterilization water control part 3, and the sterilization water control part 3 controls the temperature of the sterilization water based on the obtained temperature information. The sterilization water fed from the sterilization device 2 to the separation membrane module 1 is discharged out of the system of the separation membrane module 1 through a sterilization water discharge line 7. Hereinafter, in the separation membrane module 1, a side contacting raw liquid being a process target is referred to as the upstream, and a side contacting filtrate after the process is referred to as a downstream.

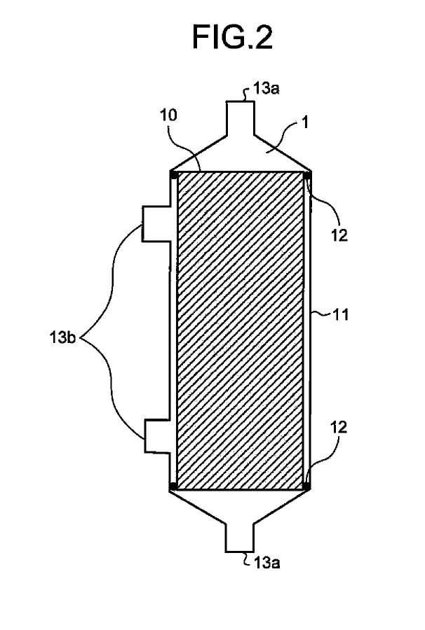

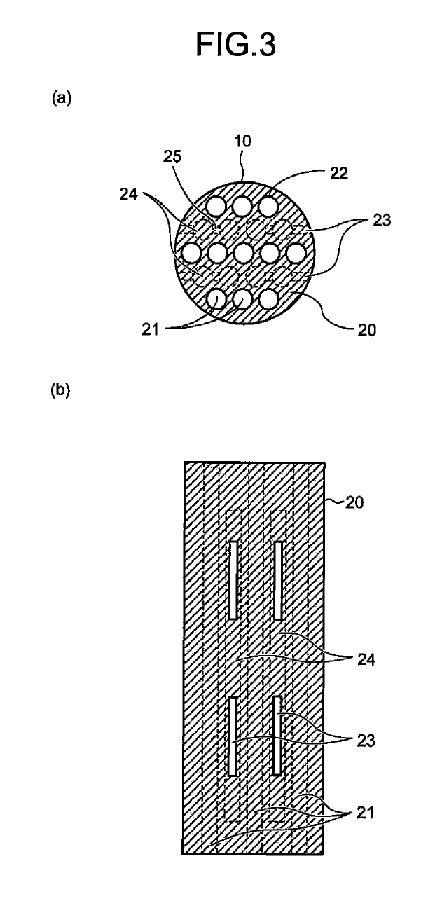

Subsequently, the separation membrane module 1 used in this the first example will be described with reference to FIG. 2 and FIGS. 3A and 3B. FIG. 2 is a view illustrating an aspect of the separation membrane module 1 used in the first example. FIG. 3A is a cross-sectional view and FIG. 3B is a side view of a monolith membrane in the separation membrane module 1 of FIG. 2.

As illustrated in FIG. 2, the separation membrane module 1 includes a ceramic membrane 10 and a module container 11. In the separation membrane module 1, a sealing member exemplified as an O-ring 12 is placed so that the upstream and the downstream of the ceramic membrane 10 are separated in an air tight and liquid tight manner. The module container 11 includes a raw liquid feed opening/concentrated liquid discharge opening 13a that feeds the raw liquid being processed or discharges the raw liquid in which filtrate is discharged and concentrated in the separation membrane module 1 and a filtrate discharge opening/backwash liquid feed opening 13b that discharges the filtrate filtrated in the separation membrane module 1 or feeds the backwash liquid cleaning the ceramic membrane 10. At least one 13a and at least one 13b are required to be provided. However, to carry out cross flow filtration that is advantageous for processing high turbidity liquid, two 13a's are required to be provided.

One or more ceramic membranes 10 are packed in one separation membrane module 1. An inner diameter of the module container 11 may be determined based on the number of the packed ceramic membranes 10. However, considering the weight and ease of handling of the module container 11, it can be said that the inner diameter is preferably 300 mm or less. When a plurality of the ceramic membranes 10 are packed, a separator or a perforated plate may be adequately provided and used in order to avoid contact of the ceramic membranes 10 with each other.

The module container 11 of the separation membrane module 1 is preferably made from a material that can endure repeated sterilization processes, that is, contact to high temperature water or vapor. Stainless steel, resins having hot-water resistance, and inorganic materials are exemplified.

Liquid tight sealing is essential for the upstream and the downstream of the separation membrane module 1, and each of the ceramic membranes 10 is required to be surely sealed in the module container 11. A method of sealing is not particularly limited. For example, a method of sealing by using a sealant and a method of sealing by directly placing the O-ring 12 between the ceramic membrane 10 and the module container 11 may be considered. When a plurality of ceramic membranes 10 are packed in the separation membrane module 1, a method of sealing by placing a perforated plate (not illustrated) in the separation membrane module 1, and then placing the O-ring 12 between the perforated plate and the ceramic membrane 10 may be considered.

When a sealant is used, an organic-based adhesive such as an epoxy resin and a urethane resin and an inorganic adhesive such as a ceramic-based adhesive are exemplified. Among them, the inorganic adhesive is preferably used because the adhesive has highly excellent heat resistance and also has excellent adhesion properties to the ceramic membrane 10. The inorganic adhesive is an adhesive in which additives such as alumina, zirconia, and magnesia are added to a paste vitrified by heating. Difference in coefficients of thermal expansion between the members can be reduced by selecting adhesive having compositions close to the compositions of the ceramic membrane. This reduces possibility of adhesion peeling between the ceramic membrane 10 and the module container 11 caused by thermal expansion and contraction associated with heating and cooling by the sterilization water. This makes the inorganic adhesive preferable as means for adhering the ceramic membrane 10 and the module container 11.

When the O-ring 12 is used, it is important that the material of the O-ring 12 has heat resistance in the range of sterilization temperature. The sterilization temperature of the separation membrane module 1 is usually 121° C. or more. Silicone rubber, fluororubber, ethylene-propylene rubber (EPM), and ethylene-propylene-diene rubber (EPDM) can be exemplified as preferable materials. When the sterilization water is desired to penetrate the ceramic membrane 10 as a gas phase, the sterilization temperature may become higher and a pressure may become higher. In this case, the material for the O-ring 12 is required to be selected with consideration of these conditions.

Subsequently, the ceramic membrane 10 used as a separation membrane will be described in detail.

Definition of ceramics is a material containing metal oxides and baked by a heat treatment at high temperature. As the metal oxides, alumina, magnesia, titanic, and zirconia can be exemplified. The separation membrane may be formed from only the metal oxides, or may include silica, silicon carbide, mullite and cordierite being compounds of silica and metal oxides. Components forming the separation membrane in addition to the ceramic are not particularly limited as long as the components can form a porous material used as the separation membrane. Examples of the components may include a metal, a resin, and a glass. However, sintering conditions include higher temperature than the melting points of almost all resins. Consequently, the metal and the glass are preferably used.

The ceramic membrane 10 in the separation membrane module 1 used in the first example is a monolith membrane illustrated in FIG. 2 and FIG. 3. Packing, efficiency of the ceramic membranes 10 into the module container 11 is improved by using the monolith membrane as the ceramic membrane 10. As the ceramic membrane 10, a flat sheet membrane, a tubular type membrane, and the like can be used in addition to the monolith membrane.

In the ceramic membrane 10 being the monolith membrane, a plurality of through-bores 21 are provided in a longitudinal direction of a ceramic substrate 20 containing at least ceramic. By forming the monolith membrane having the structure as described above, a passage area per monolith membrane can be larger. This is advantageous in both of ease of formation of the separation membrane module 1 and securement of the passage area.

The ceramic membrane 10 made by stacking separation function layers 22 on the end surface of the ceramic substrate 20 and the surface of the through-bore 21 is also preferable. A surface pore size of the ceramic membrane 10 can be more precisely controlled by stacking the separation function layers 22, and thus substances that should be filtrated can be more precisely filtrated and separated. By stacking the separation function layers 22 also on the end surface of the ceramic substrate 20, occurrence of problems that substances that should not be filtrated are filtrated through the end surface of the monolith membrane and are mixed in the filtrate can be prevented.

One or more corrective slits 23 are included in the monolith membrane. The corrective slit 23 and a corrective bore 24 communicating with the corrective slit 23 may be formed to be plugged so that raw liquid is not entered from the end face of the ceramic substrate 20. At this time, the raw liquid to be processed passes through the through-bore 21 of the monolith membrane, filtrated through the separation function layers 22, and corrected into a corrective bore 24 as the filtrate. The corrective bore 24 in the monolith membrane is communicated with the external corrective slit 23 through a corrective slit communication bore 25. The filtrate was corrected from the corrective bore 24 to the downstream of the separation membrane module 1 through the corrective slit 23. The separation membrane area per unit area decreases by plugging a part of the through-bores in the end part of the monolith membrane. However, resistance of flowing liquid can be reduced because a path of this separation membrane where the filtrate is filtrated from each through-bore to the downstream through the separation film is shorter than a path of a monolith membrane having no slits. A ratio of the number of the through-bores 21 to the number of the corrective bores 24 is not particularly limited.

A porosity of the ceramic membrane 10 is not particularly limited. However, a too low porosity decreases filtrate efficiency and a too high porosity deteriorates strength. The preferable porosity is 20% or more and 60% or less in order to satisfy both filtration efficiency and strength of the separation membrane and to provide durability that repeated sterilization can be carried out.

Here, the porosity is determined by the following formula.

Porosity [%]=(100×(Wet membrane weight [g]−Dry membrane weight [g]))/(Specific gravity of water [g/cm³]×Membrane volume [cm³])

Here, the wet membrane means a membrane in a state that the pores are filled with pure water, but pure water is not entered into the hollow part. The dry membrane means a membrane in a state that pure water is not contained in the pores. The membrane volume can be calculated by subtracting a volume where the hollow part occupies from a volume where separation membrane occupies.

An adequate value of pure water permeability performance of the ceramic membrane 10 can be determined from a required filtrate amount and properties of processing raw liquid. An adequate value of an average pore diameter of the ceramic membrane 10 also can be determined from the properties of processing raw liquid and required properties of the filtrate.

The ceramic membrane 10 used in the first embodiment is a membrane made by staking one layer of the separation function layer 22 on the ceramic substrate 20. However, two or more layers of the separation function layers 22 may be stacked. By stacking two or more layers of the separation function layers having different pore diameters, the average pore diameter in the whole ceramic membrane 10 can be adjusted and fouling resistance also can be improved by providing a hydrophilic layer on the surface of the separation membrane. The thickness of the separation function layers 22 is not particularly limited. However, when the thickness is less than 1 μm, it is not preferable because the strength is insufficient, whereas when the thickness exceeds 200 μm, it is not preferable because the water permeability performance deteriorates. For this reason, when the separation function layers 22 is provided, the thickness thereof is preferably 1 μm or more and 200 μm or less. The separation function layers 22 of the ceramic membrane 10 are preferably made of alumina, magnesia, titania, and zirconia as described above. Particularly, titania can be preferably used because titania particularly has excellent fouling resistance.

Considering ease of production, a shape of a cross section perpendicular to the longitudinal direction of the ceramic substrate 20 of the monolith membrane is preferably a circular shape or a polygonal shape. In the case of the polygonal shape, regular polygons are particularly preferable from the viewpoint of packing efficiency in the module. Among the regular polygons, regular polygons such as regular triangle, square, and regular hexagon that can be paved without gap by one type of figure are further preferable. The outer diameter of the ceramic substrate 20 is preferably 10 mm or more and 300 mm or less, more preferably 20 mm or more and 250 mm or less, and further preferably 30 mm or more and 200 mm or less. When the shape of the ceramic substrate 20 is a polygonal column, the outer diameter is determined as a diameter of circumcircle of the end face where the end face is triangle, and the outer diameter is determined as the longest length of segments that are formed by connecting any two apices where the end face is a polygonal shape other than triangle. The number of the through-bores becomes small when the outer diameter of the ceramic substrate 20 is less than 10 mm, whereas the production becomes difficult when the outer diameter exceeds 300 mm. The length of the ceramic substrate 20 in a longitudinal direction is preferably 20 mm or more and 2000 mm or less, more preferably 30 mm or more and 1700 mm or less, and further preferably 40 mm or more and 1500 mm or less. A membrane area per ceramic membrane 10 becomes small when the length in the longitudinal direction of the ceramic substrate 20 is less than 20 mm, whereas the production and the handling become difficult when the length exceeds 2000 mm.

Considering the membrane area and strength per unit volume, the number of the through-bores 21 provided in the ceramic substrate 20 of the monolith membrane (including the number of the corrective bores 24) is preferably 10 or more and 5000 or less, and more preferably 30 or more and 2000 or less. The number of the through-bores 21 exceeding 5000 is not preferable because the production becomes difficult and the strength decreases.

The shape of the through-bore 21 can be preferably selected from shapes such as circle, oval, a polygonal shape, and a star shape. An equivalent diameter of the shape is preferably 0.5 mm or more and 5 mm or less. In the present invention, the equivalent diameter of the through-bore 21 means an inside diameter (internal diameter) when the cross section of the through-bore 21 is circle, or a diameter of a circle formed by drawing a circle having the same area as the cross section when the cross section of the through-bore 21 is not circle.

When a filtration process is carried out using the separation membrane module 1 as described above, a sterilization process by the method for sterilizing separation membrane module according to the first example can be carried out before starting the filtration process or at any stage during the filtration process. When the sterilization of the separation membrane module during the filtration process is carried out, it is preferable that the sterilization is carried out after stopping the feed of the processing raw liquid and cleaning inside of the separation membrane module. Sterilization water may be used for the cleaning of the separation membrane module. Temperature of the sterilization water is preferably controlled by the method for controlling the temperature described below.

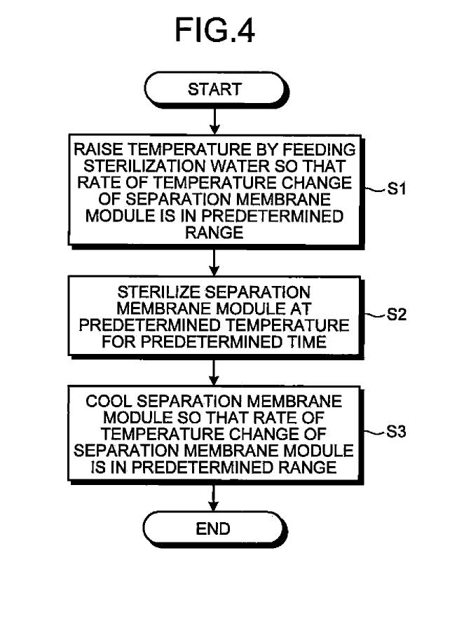

The method for sterilizing separation membrane module 1 according to the first example will be described with reference to FIG. 4. FIG. 4 is a flowchart illustrating a sterilization process of the separation membrane module 1 according to the first example.

In the method for sterilizing the separation membrane module 1 according to the first example, the method includes raising the temperature of the ceramic membrane 10 of the separation membrane module 1 to a predetermined sterilization temperature by feeding sterilization water having a controlled temperature and pressure by the sterilization water control part 3 into the upstream of the separation membrane module 1 so that a rate of temperature change ΔT1 at the upstream of the separation membrane module 1 is 6.0° C. or less per minute (Step S1). We found that a reduction effect in deterioration of the ceramic membrane 10 is high when the rate of temperature change ΔT1 is 6.0° C. or less per minute. The damage of the ceramic membrane 10 in the separation membrane module 1 can be reduced by feed backing temperature information to the sterilization water control part 3 and controlling the temperature of the sterilization water so that ΔT1 is 6.0° C. or less, with measuring a temperature T1 at the upstream of the separation membrane module 1 with the temperature measurement part 5.

The lower limit of the rate of temperature change ΔT1 is not particularly limited. However, problems of too much time for sterilization of the ceramic membrane 10 and difficulty of ΔT1 control may be considered, when ΔT1 is too small. For this reason, it is preferable that the temperature of the sterilization water is controlled in the sterilization water control part 3 so that ΔT1 is 0.01° C. or more per minute.

ΔT1 at the time of temperature raise (and temperature drop) may be controlled to be constant or to vary. When ΔT1 is varied, an instantaneous rate of temperature change of the separation membrane module 1 is required to be controlled to be equivalent to 6.0° C. or less per minute.

When the temperature of the sterilization water in the separation membrane module 1 becomes 100° C. or more, positive pressure state in the separation membrane module 1 is required. At this time, as a method of managing pressure in the separation membrane module 1, a method including providing a pressure measurement part in the sterilization water feed line 4, sending the pressure data measured by the pressure measurement part to the sterilization water control part 3, and feed-backing to pressure control at the sterilization water control part 3 is preferably used.

The sterilization water fed to the separation membrane module 1 is a state of either a liquid phase or a gas phase and means water that has a controlled temperature and pressure and is sterilized. Ion-exchanged water, reverse osmosis membrane permeated water, distilled water, and water having cleanliness similar to these types of water are preferably used for the sterilization water.

The sterilization water control part 3 makes the sterilization water in the state of the liquid phase or the gas phase and controls the sterilization water in a predetermined temperature and pressure. The sterilization water may be obtained by a process in which ion-exchanged water, reverse osmosis membrane permeated water, distilled water, or the like is previously sterilized, and thereafter, water of the liquid phase or gas phase having the predetermined temperature and pressure is formed, or a process in which ion-exchanged water, reverse osmosis membrane permeated water, distilled water, or the like is previously treated so as to form water of the liquid phase or a gas phase having the predetermined temperature and pressure, and thereafter, the water is sterilized through a sterilization filter or the like.

As the sterilization water control part 3, water may be heated with a heater or a widely known boiler can be used. As a method of controlling a temperature and a pressure, a function may be added to the boiler, or a heat exchanger, a compressor, a pressure pump, and the like may be separately provided. With regard to the temperature control, a controller that can be applied for not only a heating process but also a cooling process is preferably used.

The sterilization water having a controlled temperature and pressure by the sterilization water control part 3 is fed to the upstream of the separation membrane module 1 through the sterilization water feed line 4. In the first example, the separation membrane module 1 is vertically placed. The sterilization device 2 is located on the upper part of the separation membrane module 1, and the sterilization water is fed from the upper part. When the sterilization water is fed to the separation membrane module 1 mainly in a state of the gas phase, drain generated by condensation of the sterilization water or the like is discharged in a vertically downward direction, and thus, retention of drain is difficult to occur in the separation membrane module 1 by feeding the sterilization water from the upper part as described in the first embodiment. This can prevent sterilization failure.

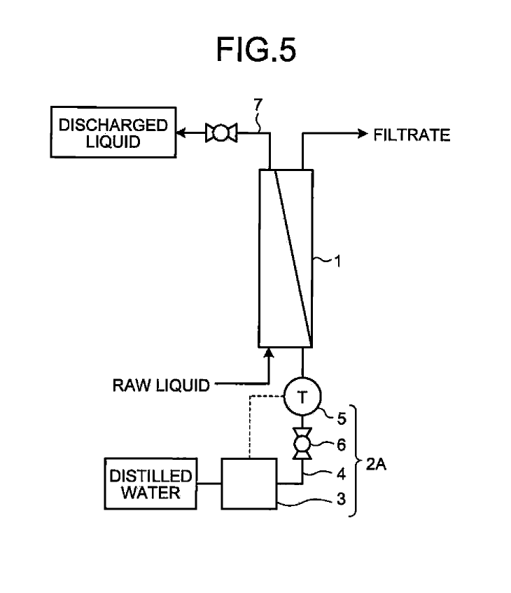

On the other hand, when the sterilization water is fed to the separation membrane module 1 as the liquid phase, the gas existing in the separation membrane module 1 can be exhausted to an upper part by locating the sterilization device 2 to feed the sterilization water from an under part of the separation membrane module 1 as illustrated in FIG. 5, and thus retention of the gas is difficult to occur in the separation membrane module 1. This is preferable because sterilization failure is difficult to occur.

After the temperature of the separation membrane module 1 reaches a predetermined sterilization temperature, the separation membrane module 1 is sterilized in a predetermined temperature and predetermined time (Step S2). In sterilization using water vapor, usually, sterilization temperature is 121° C. and sterilization time is 15 minutes to 20 minutes. However, the sterilization temperature and the sterilization time can be adequately changed depending on a level of the sterilization required for the separation membrane module 1. Preferably, the sterilization water is continuously fed to the separation membrane module 1 to easily maintain the temperature. However, if sterilization conditions can be satisfied, the sterilization process may be carried out with stopping the feed of the sterilization water.

After completion of the sterilization process, a temperature of the ceramic membrane 10 in the separation membrane module 1 is cooled to the predetermined temperature with controlling so that ΔT1 is 6.0° C. or less per minute (Step S3). To shorten the cooling time of the separation membrane module, it is preferable that T1 is decreased so as to be 6.0° C. or less per minute by feeding to the upstream of the separation membrane module 1 the sterilization water having a controlled temperature and pressure by the sterilization water control part 3.

As described above, moderate temperature change in the separation membrane module 1 is maintained by carrying out the sterilization process of the separation membrane module 1, and thus, the damage of the ceramic membrane 10 associated with rapid temperature change can be reduced.

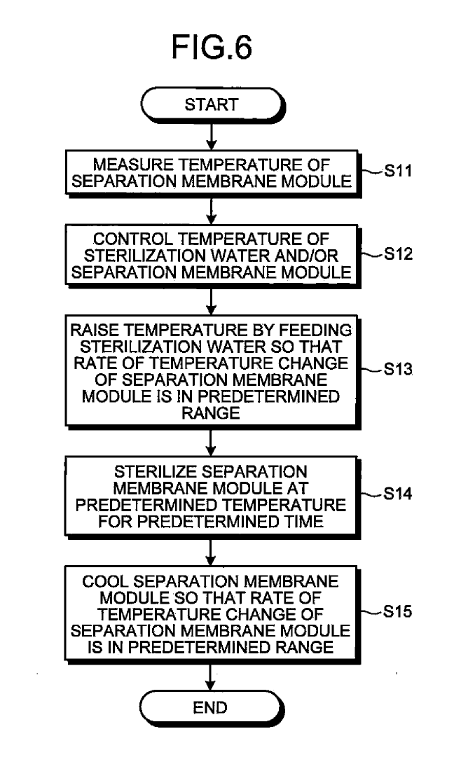

On the other hand, when the separation membrane module 1 after using the filtration process is sterilized, the separation membrane module 1 is preferably sterilized after cleaning because suspending solid and the like may be attached on the surface and in the pores. At this time, the cleaning of the separation membrane module 1 may be carried out, for example, using a cleaning liquid of about 80° C. Just after this cleaning, when sterilization water having a normal temperature (20° C. to 30° C.) is fed into the separation membrane module 1, the ceramic membrane 10 is subjected to rapid temperature change and may cause damage by feeding the sterilization water because the temperature of the separation membrane module has similar temperature to the temperature of the cleaning liquid. In this case, the temperature of sterilization water at the time of starting the feed should be controlled. Hereinafter, a method of sterilizing a separation membrane module 1 according to a second modification of the first example will be described with reference to FIG. 6. FIG. 6 is a flowchart illustrating a sterilization process of the separation membrane module 1 according to the second modification of the first example.

In the method of sterilizing the separation membrane module 1 according to the second modification, first, a temperature T1 of the upstream of the separation membrane module 1 is measured (Step S11). To measure the temperature T1 of the upstream of the separation membrane module, the temperature measurement part 5 may be provided to communicate with the upstream of the separation membrane module 1. However, when the temperature measurement part 5 is provided so that the temperature measurement part 5 is in contact with the upstream of the separation membrane module 1, the temperature of the upstream of the ceramic membrane 10 can be measured. This is preferable because the temperature acts as a high accuracy index for reducing the damage of the ceramic membrane 10.

Subsequently, a temperature Tw of sterilization water and/or temperature T1 of the upstream of the separation membrane module 1 are controlled so that absolute temperature difference |T1−Tw| between the temperature Tw of the sterilization water fed to the separation membrane module 1 and the temperature T1 of the upstream of the separation membrane module 1 measured in the previous temperature measurement process is 30.0° C. or less (Step S12). By this process, temperature difference when the sterilization water and the ceramic membrane 10 in the separation membrane module 1 are contacted becomes small, and thus, the damage caused by rapid temperature change of the ceramic membrane 10 can be reduced.

A preferable range of |T1−Tw| is adequately determined based on various factors such as a size of the separation membrane module, a volume of sterilization target sections, a material of the ceramic membrane 10, and porosity. When the separation membrane module 1 including a generally used ceramic membrane 10 is used, |T1−Tw| may be 30.0° C. or less. |T1−Tw| is preferably 20.0° C. or less, and |T1−Tw| is more preferably 10.0° C. or less. The best case of reducing damage risk of the ceramic membrane 10 is that |T1−Tw| is equal to 0° C. When control of |T1−Tw| to be 0° C. is difficult, |T1−Tw| may be restricted within the range of the above-described values.

It is preferable that |T1−Tw| is set to 30.0° C. or less by controlling the temperature Tw of the sterilization water because the temperature Tw of the sterilization water can be controlled by the sterilization water control part 3. However, |T1−Tw| is set to 30.0° C. or less by controlling the surrounding temperature of the separation membrane module 1.

The sterilization water controlled to the predetermined temperature Tw by the sterilization water control part 3 is fed to the upstream of the separation membrane module 1 through the sterilization water feed line 4.

After the difference between the temperature Tw of the sterilization water and the temperature T1 of the upstream of the separation membrane module 1, that is |T1−Tw|, becomes 30.0° C. or less, feed of the sterilization water to the separation membrane module 1 is started and the temperature of the ceramic membrane 10 of the separation membrane module 1 is raised to the predetermined sterilization temperature by feeding to the upstream of the separation membrane module 1 the sterilization water in which a temperature and a pressure are controlled by the sterilization water control part 3 so that the rate of temperature change ΔT1 is 6.0° C. or less per minute (Step S13). After temperature of the separation membrane module 1 reaches the predetermined sterilization temperature, the separation membrane module 1 is sterilized at the predetermined temperature for the predetermined time (Step S14).

After completion of the sterilization process, the temperature of the ceramic membrane 10 in the separation membrane module 1 is cooled to the predetermined temperature so that ΔT1 is 6.0° C. or less per minute (Step S15).

Moderate temperature change in the separation membrane module 1 is maintained by carrying out the sterilization process of the separation membrane module 1 as described above, and thus, the damage of the ceramic membrane 10 associated with rapid temperature change can be reduced.

In the sterilization process described above, the sterilization water fed to the upstream of the separation membrane module 1 is discharged from the sterilization water discharge line 7. In the first example, to feed the sterilization water in the gas phase, the sterilization device 2 is located on the upper part of the separation membrane module 1 and the sterilization water discharge line 7 is located under the lower part of the separation membrane module 1. When the sterilization water is fed in the liquid phase, as illustrated in FIG. 5, a sterilization device 2A is located under the lower part of the separation membrane module 1 and the sterilization water discharge line 7 is located on the upper part of the separation membrane module 1. This is preferable because both of reduction in sterilization failure and improvement of discharge efficiency of the sterilization water are satisfied.

When the sterilization water is discharged from the separation membrane module 1, the sterilization water may be returned to the sterilization water control part 3 again. It is preferable that because the sterilization water is continuously fed to the separation membrane module 1, and thus, energy required for controlling a temperature and a pressure of the sterilization water can be reduced by returning the sterilization water to the sterilization water control part 3. To return the sterilization water, the sterilization water discharge line 7 of the separation membrane module 1 and the sterilization water control part 3 are connected through a sterilization water return line (not illustrated). This enables reuse of the sterilization water.

When the sterilization water is returned, the sterilization water may be returned to the sterilization water control part 3 after removing scale of the sterilization water by providing a filter in midstream of the sterilization water return line. Both of the sterilization water discharge line 7 and the sterilization water return line may be provide by assuming high turbidity of the discharged sterilization water. In this case, when the discharged sterilization water has high turbidity, the sterilization water is discharged out of the system through the sterilization water discharge line 7, and when the discharged sterilization water has low turbidity, the sterilization water may be returned to the sterilization water control part 3 through the sterilization water return line. A procedure including measuring the turbidity of the discharged sterilization water by a turbidity meter; feed backing the obtained turbidity data to an electromagnetic valve; and switching the sterilization water discharge line and the sterilization water return line by opening and closing the electromagnetic valve is also preferably used.

In the first example, the temperature T1 of the upstream of the separation membrane module 1 is measured. When T1 cannot be directly measured, however, a procedure including preparing a ceramic membrane 10 having the same specification as the measurement target, previously examining a correlation between T1 and a temperature To at any point on the outer surface of the separation membrane module 1, and, at the time of the actual sterilization process, measuring To, and reversely calculating T1 from the correlation may be used. As another procedure, the separation membrane module 1 is controlled in a constant temperature by flowing water previously controlled at the constant temperature through the separation membrane module 1, and the temperature of the constant temperature water may be presumed as the temperature T of the separation membrane module 1.

### Second Example

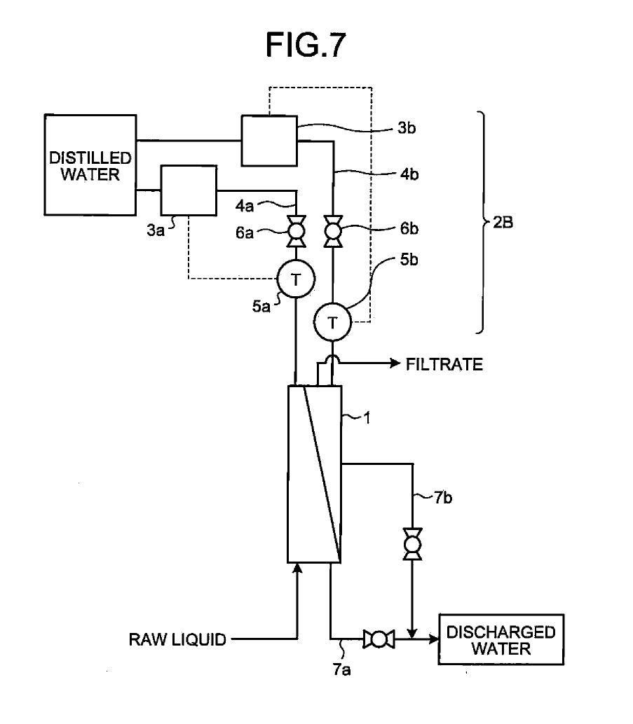

A sterilization device according a second example has two sterilization water feed lines, and is different from the sterilization device according the first example in that sterilization water is fed to the upstream and the downstream of a separation membrane module through the two sterilization water feed lines. Hereinafter, the second example will be described with reference to the drawings. FIG. 7 is a schematic view of the sterilization device of the separation membrane module according to the second example.

This sterilization device 2B includes sterilization water control parts 3a and 3b feeding the sterilization water having a controlled temperature and pressure to each of the upstream and the downstream of the separation membrane module 1; temperature measurement parts 5a and 5b measuring temperatures of the upstream and the downstream of the separation membrane module 1; and sterilization water feed lines 4a and 4b of the upstream and the downstream of the separation membrane module 1.

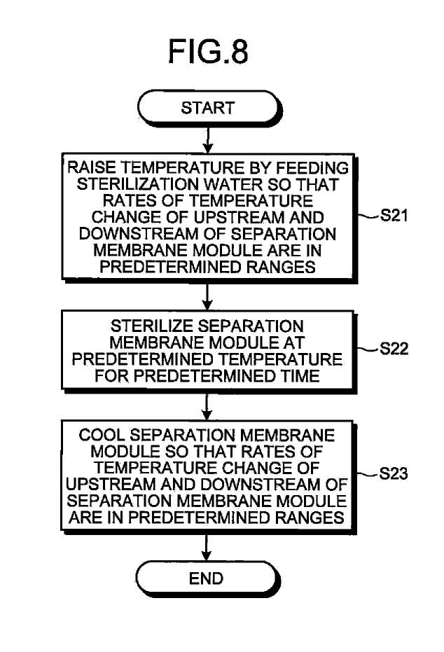

A method of sterilizing the separation membrane module 1 according to the second example will be described with reference to FIG. 8. FIG. 8 is a flowchart illustrating a sterilization process of the separation membrane module 1 according to the second example.

In the second example, the method includes raising the temperature of the ceramic membrane 10 in the separation membrane module 1 to a predetermined sterilization temperature by feeding the sterilization water having a controlled temperature and pressure by the sterilization water control parts 3a and 3b into the upstream and the downstream of the separation membrane module 1 so that a rate of temperature change ΔT1 at the upstream and a temperature change ΔT2 at the downstream of the separation membrane module 1 is 6.0° C. or less per minute (Step S21). ΔT1 and ΔT2 of the ceramic membrane 10 may be independently controlled. However, for the temperature T1 of the upstream and the temperature T2 of the downstream of the ceramic membrane 10, it is preferable that each ΔT1 and ΔT2 is controlled so that |T1−T2| becomes close to 0.

Concerns about deterioration and damage of the ceramic membrane 10 can be reduced by maintaining moderate temperature change of the ceramic membrane 10 in the separation membrane module 1 after the feed of the sterilization water to the separation membrane module 1 starts.

After the temperature of the separation membrane module 1 reaches the predetermined sterilization temperature, the separation membrane module 1 is sterilized at the predetermined temperature for a predetermined time (Step S22). To maintain the separation membrane module 1 at the predetermined temperature, it is preferable that the sterilization process is carried out by feeding the sterilization water having the predetermined temperature and the predetermined pressure to the upstream and the downstream of the separation membrane module 1.

After completion of the sterilization process, the temperature of the ceramic membrane 10 in the separation membrane module 1 is cooled to the predetermined temperature so that ΔT1 and ΔT2 of the separation membrane module are 6.0° C. or less per minute (Step S23). To shorten the cooling time, it is preferable that the separation membrane module 1 is cooled by feeding the sterilization water with a temperature and a pressure controlled by the sterilization water control parts 3a and 3b to the upstream and the downstream of the separation membrane module 1.

In the second example, concerns about the damage of the ceramic membrane 10 can be further reduced by feeding the sterilization water to each of the upstream and the downstream of the separation membrane module and sterilizing with controlling the temperature of the whole ceramic membrane 10 closer to uniform temperature.

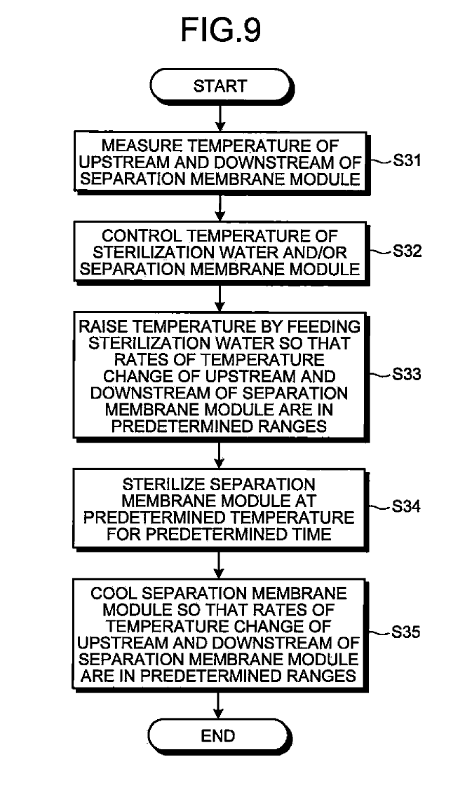

It is also preferable that the sterilization process is carried out by controlling the temperature of the sterilization water at the time of starting the feed of the sterilization water fed to the upstream and the downstream of the separation membrane module 1. FIG. 9 is a flowchart illustrating a sterilization process of the separation membrane module 1 according to a first modification of the second example.

In the first modification, temperatures T1 and T2 of the upstream and the downstream of the separation membrane module 1 are measured (Step S31). A temperature Tw1 of the upstream of the separation membrane module 1 and a temperature Tw2 of sterilization water feeding to the downstream of the separation membrane module 1 and/or the temperature T1 of the upstream of the separation membrane module 1 and the temperature T2 of the downstream of the separation membrane module 1 are controlled so that both absolute temperature difference |T1−Tw1| between the temperature Tw1 and the temperature T1 of the upstream of the separation membrane module 1 and absolute temperature difference |T2−Tw2| between the temperature Tw2 and the temperature T2 of the downstream of the separation membrane module 1 are 30.0° C. or less (Step S32). By this process, temperature difference at the time of contacting the sterilization water and the ceramic membrane 10 in the separation membrane module 1 becomes small, and thus, damage caused by the rapid temperature change of the ceramic membrane 10 can be reduced.

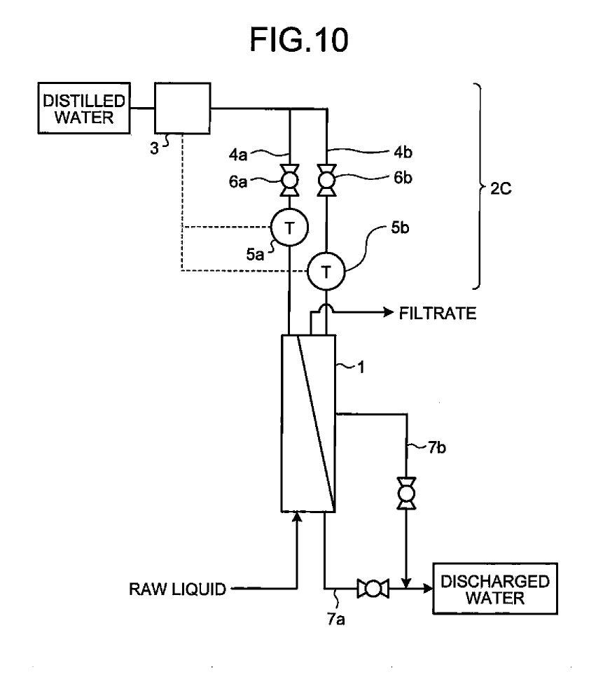

It is preferable that to reduce the temperature difference between the upstream and the downstream of the separation membrane module 1, Tw1 and Tw2 at the time of starting sterilization and during the feed of the sterilization water to the separation membrane module are controlled so as to be closer values as possible, or a rate of temperature change ΔT1 of T1 and a rate of temperature change ΔT2 of T2 are controlled so as to be closer values as possible. As illustrated in FIG. 10, it is further preferable that, when the sterilization water control part 3 is commonly used at the upstream and the downstream of the separation membrane module, not only dose Tw1 equal to Tw2 but also the effect of reducing the cost for controlling a temperature and a pressure of the sterilization water is provided. FIG. 10 is a schematic view of a sterilization device of a separation membrane module according to a second modification of the second example. In a sterilization device 2C, a similar effect also can be obtained in a manner that T1 or T2 of the separation membrane module 1 is measured by any one of temperature measurement parts 5a and 5b, and sterilization water is fed after controlling |T1−Tw1| or |T2−Tw2| being 30.0° C. or less.

The sterilization water controlled at the predetermined temperatures Tw1 and Tw2 by the sterilization water control parts 3a and 3b is fed to the upstream and the downstream of the separation membrane module 1 through the sterilization water feed lines 4a and 4b and valves 6a and 6b, respectively

After the temperatures Tw1 and Tw2 of the sterilization water and the temperatures T1 and/or T2 of the upstream of the separation membrane module 1 are |T1−Tw1|≦30.0° C. and/or |T1−Tw2|≦30.0° C., the sterilization water is started to be fed to the separation membrane module 1, and the temperature of the ceramic membrane 10 in the separation membrane module 1 is raised to a predetermined sterilization temperature by feeding to the upstream and the downstream of the separation membrane module 1 the sterilization water having a controlled temperature and pressure by the sterilization water control parts 3a and 3b so that ΔT1 and ΔT2 are 6.0° C. or less per minute (Step S33)

After the separation membrane module 1 reaches the predetermined sterilization temperature, the separation membrane module 1 is sterilized at predetermined temperature for a predetermined time (Step S34). After completion of the sterilization process, the temperature of the ceramic membrane 10 in the separation membrane module 1 is cooled to the predetermined temperature so that ΔT1 and ΔT2 are 6.0° C. or less per minute (Step S35).

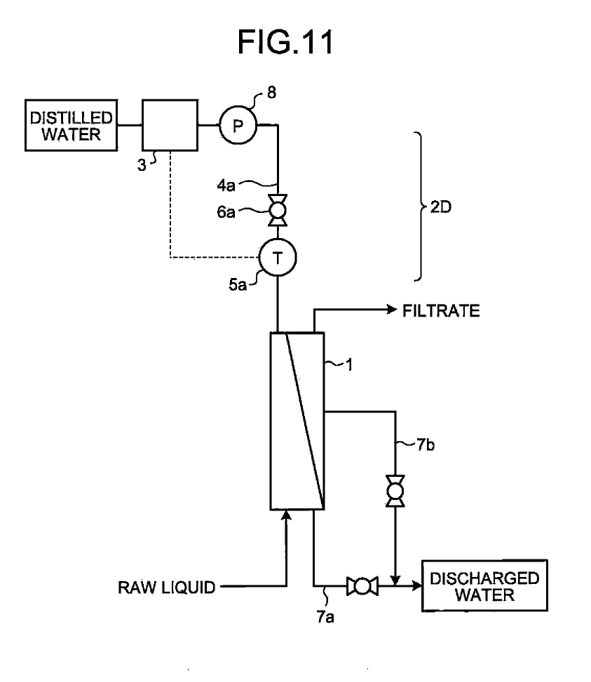

As another method for feeding the sterilization water to the upstream and the downstream of the separation membrane module, after the sterilization water is fed to the upstream or the downstream, the sterilization water may permeate the ceramic membrane 10. FIG. 11 is a schematic view of a sterilization device of a separation membrane module according to a third modification of the second example.

In the third modification in the second example, this sterilization device 2D feeds the sterilization water having an adjusted temperature and pressure to the upstream of the separation membrane module 1. At the upstream and the downstream of the separation membrane module 1, sterilization water discharge lines 7a and 7b are provided, respectively. On the sterilization water feed line 4, a sterilization water feed pump 8 is located between the sterilization water control part 3 and the valve 6.

When the sterilization water is fed from the sterilization device 2D to the upstream of the separation membrane module 1, the sterilization water is discharged out of the system through the sterilization water discharge line 7a. By driving the sterilization water feed pump 8, the sterilization water fed to the upstream is pressurized and the sterilization water is filtrated to the downstream by the pressure. The sterilization water filtrated to the downstream is discharged out of the system through the sterilization water discharge line 7b.

In the third modification, the sterilization water is distributed even inside of the pores of the ceramic membrane 10 by permeating the ceramic membrane 10 with sterilization water. This further improves sterilization efficiency. As a method of permeating the ceramic membrane 10 with the sterilization water, in addition to the method for pressurizing the sterilization water using the sterilization water feed pump 8 as described above, a method of locating a suction pump in the sterilization water discharge line 7b discharging the sterilization water from the downstream and permeating to the downstream by sucking the sterilization water with the suction pump is exemplified.

At this time, there is difference in permeablity of the ceramic membrane 10 when the sterilization water is the liquid phase or the gas phase. In the case of the gas phase, higher pressurization is required. Some difference in the operation is generated in the case of the liquid phase or the gas phase. The operation in each case will be sequentially described. As a matter of convenience, the sterilization water is determined to be fed to the upstream of the separation membrane module 1. When T2 of the separation membrane module 1 is measured and the sterilization water is fed to the downstream of the separation membrane module 1, it can be considered that the upstream and the downstream in the following case are replaced.

When the sterilization water is liquid phase, the sterilization water may be fed so as to apply required pressure for filtration. In this case, at least temperature T1 of the upstream of the separation membrane module 1 is measured and Tw is controlled so as to be |T1−Tw|≦30.0° C. before feeding the sterilization water, and thereafter, the sterilization water is fed to the upstream of the separation membrane module 1.

The sterilization water permeates the ceramic membrane 10 by pressurizing from the upstream and/or sucking from the downstream with feeding the sterilization water to the upstream of the separation membrane module 1. A rate of temperature change and discharge of the sterilization water of the downstream of the separation membrane module 1 are in accordance with the method for sterilizing described above.

After the separation membrane module 1 reaches a predetermined temperature, to make it easier to maintain the temperature in a process of sterilization by maintaining temperature, it is preferable that the sterilization water is fed to the upstream and the fed sterilization water continuously permeates to the downstream. A temperature drop process after maintaining for a predetermined time is also in accordance with the method of sterilizing described above.

When the sterilization water is the gas phase, the gas phase sterilization water may be used in either of a saturated state or an unsaturated state. For reference, when an average pore diameter of an alumina ceramic membrane is 0.2 μm, sterilization water permeates the separation membrane module 1 from the upstream to the downstream by applying a pressure of about 400 kPa. This pressure is corresponding to saturated water vapor pressure of a water vapor of about 145° C.

When the sterilization water that is the gas phase and the saturated state permeates the ceramic membrane 10, the temperature of the ceramic membrane 10 is raised with previously feeding the sterilization water to the upstream and the downstream of the separation membrane module 1. Both of the gas phase and the liquid phase are applicable for the sterilization water fed for the temperature rise. However, the gas phase sterilization water is preferably fed because an aspect of preferable piping is different from the gas phase and the liquid phase. At this time, control of an initial temperature and a rate of temperature change of the sterilization water is carried out in accordance with the method of sterilizing by feeding the sterilization water to both of the upstream and the downstream described above.

After temperature of the ceramic membrane 10 is sufficiently raised and pressure of the sterilization water is sufficient for permeation of the ceramic membrane, the sterilization water is fed only to the upstream of the separation membrane module 1 and the sterilization water may permeate the ceramic membrane 10 from the upstream to the downstream. Continuous permeation of the sterilization water is preferable to maintain the temperature. Similar to the temperature raise step, a temperature drop step is carried out in accordance with the method for sterilizing by feeding the sterilization water to both of the upstream and the downstream.

### Third Example

Subsequently, an apparatus for producing chemicals by continuous fermentation according to a third example will be described. FIG. 10 is a schematic view of the apparatus for producing chemicals according to the third example.

An apparatus for producing chemicals 200 includes a separation membrane module 1 including a separation membrane containing ceramic, the sterilization device 2C as referred in the first modification of the second example, a fermentation tank 100 that converts a fermentation feedstock into fermented liquid containing chemicals by fermentation culture of the feedstock by microorganisms; and a circulation pump 101 being a fermented liquid circulation means that sends the fermented liquid from the fermentation tank 100 to the separation membrane module 1. The apparatus for producing chemicals 200 is a continuous fermentation apparatus that produces chemicals by the fermentation in the fermentation tank 100 and carries out continuous fermentation by refluxing non-filtrated liquid to the fermentation tank 100 with filtering the fermented liquid containing the produced chemicals by the separation membrane module 1. To the apparatus for producing chemicals 200, the sterilization device 2C that feeds the sterilization water to upper parts of each of the upstream and the downstream of the separation membrane module 1 is connected. However, the sterilization of the separation membrane module 1 also can be carried out by connecting sterilization devices 2, 2A, and 2B.

Before the continuous fermentation starts, a sterilization process of whole system of the apparatus for producing chemicals 200 including the separation membrane module 1 is required. The sterilization of the separation membrane module 1 may be carried out by the method of the second example illustrated and the other methods described above. The sterilization in the system of the apparatus for producing chemicals 200 other than the separation membrane module 1 can be carried out by steam sterilization or hot-water sterilization. At this time, it is preferable that the temperature of the ceramic membrane 10 is controlled to avoid rapid change by locating a valve (not illustrated) or the like between the separation membrane module 1 and a place other than the separation membrane module 1 to block vapor and hot water. On the apparatus for producing chemicals, the sterilization process is carried out only before starting the continuous fermentation and is not carried out during the continuous fermentation.

After the whole system of the apparatus for producing chemicals 200 is sterilized, production of the chemicals by the continuous fermentation is started. The continuous fermentation in the fermentation tank 100 is carried out with maintaining high productivity by feeding a culture medium to the fermentation tank 100 with a culture medium feed pump 107 if needed, stirring the fermented liquid in the fermentation tank 100 with a stirring device 103 if needed, feeding a neutralizing agent with a pH sensor control device 104 and a neutralizing agent feed pump 108 and adjusting, the pH of the fermented liquid if needed, and feeding adequate gas with a fermentation tank gas feed device 115 if needed.

Internal pressure in the fermentation tank 100 may rise with progress of the fermentation. When gas is fed by using the fermentation tank gas feed device 115, it is preferable that the fed gas is easy to be dissolved into the fermented liquid when the inside of the fermentation tank 100 is positive pressure. However, if the pressure becomes too high, the fermentation tank 100 is damaged. Consequently, the internal pressure is preferably controlled by a fermentation tank pressure adjustment valve 116 and a fermentation tank pressure gauge 117.

The fermented liquid in the fermentation tank 100 circulates between the separation membrane module 1 and the fermentation tank 100 by the circulation pump 101. The fermented liquid containing the chemicals is filtrated and separated into microorganism and filtrate containing the chemicals by the separation membrane module 1 and can be taken out from the apparatus for producing chemicals 200. The filtrated and separated microorganisms stay in the apparatus, and thus, a high microorganism concentration in the system can be maintained and fermentation production having high production speed can be carried out.

The filtration and separation by the separation membrane module 1 can be carried out without using particular power because of the pressure generated by the circulation pump 101. However, an amount of the fermented liquid can be adequately adjusted by providing a filtration pump 109 if needed and using a differential pressure difference sensor 106. At this time, it is important that the filtration process should be carried out in a range of a transmembrane pressure difference of 500 kPa or less. The transmembrane pressure difference means a pressure difference between the upstream and the downstream of the ceramic membrane. When the transmembrane pressure difference becomes out of the range, clogging of the microorganisms and the culture medium components is rapidly generated. This causes decrease in permeation rate and failure may occur in the continuous fermentation. Adjustment of the transmembrane pressure difference also may be carried out by sucking pressure of the filtration pump 109, and pressure control of gas or liquid introduced in the system.

If needed, a high microorganism concentration can be maintained because a temperature in the fermentation tank 100 can be maintained in a temperature that activates microorganisms/cultured cells by a temperature control device 102. In a state that the fermented liquid is flown in the separation membrane module 1, a rate of temperature change in the fermented liquid is also preferably controlled at 6.0° C. or less per minute.

Backwash piping is provided at the downstream and backwash liquid can be introduced using a backwash pump 111, if needed so that backwash of the separation membrane module 1 can be carried out. The backwash means a method of removing scale substance on the membrane surface by permeating the ceramic membrane from the upstream to the downstream with liquid. At this time, the backwash can be carried out by closing a backwash valve 112, stopping the backwash pump 111, opening a filtration valve 110, and operating the filtration pump 109 at the time of carrying out the separation membrane filtration, whereas by closing the filtration valve 110, stopping the filtration pump 109, opening the backwash valve 112, and operating the backwash pump 111 at the time of not carrying out the separation membrane filtration. Gas is fed inside of the separation membrane module 1 to carry out cleaning of clogged substance deposited on the separation membrane surface by using a piping gas feed control valve 113 and a piping scrubbing gas feed device 114. The piping gas feed control valve and the piping scrubbing gas feed device are controlled by a timer or a control device if needed, and control the feed of the scrubbing gas. Pressure difference of the separation membrane module 1 is measured by the pressure difference sensor 106 if needed, and the piping gas feed control valve can be adjusted if needed.

### (About Microorganisms Used for Continuous Fermentation)

The fermentation feedstock of microorganism and cultured cells used in the continuous fermentation, that is, a substance before conversion, may be a substance that can promote growth of the microorganisms and the cultured cells carrying out fermentation culture and can produce chemicals that are target fermentation products well. As the fermentation feedstock, for example, a common liquid culture medium adequately containing carbon sources, nitrogen sources, inorganic salts, and, if needed, organic micronutrients such as amino acids and vitamins is preferably used. For example, discharged water or sewage water also can be used without treatment or by adding a fermentation feedstock, as long as the liquid partially contains the substance that can promote growth of the microorganisms and the cultured cells carrying out the fermentation culture and can produce chemicals that are target fermentation products well.

As the carbon source described above, for example, sugars such as glucose, sucrose, fructose, galactose, and lactose, starch containing these sugars, starch hydrolysate, sweet potato syrup, sugar beet syrup, cane juice, extract or concentrated liquid of sugar beet syrup or cane juice, filtrate of sugar beet syrup or cane juice, syrup (high test molasses), raw sugar purified or crystallized from sugar beet syrup or cane juice, purified sugar purified or crystallized from sugar beet syrup or cane juice, and further organic acids such as acetic acid and fumaric acid, alcohols such as ethanol, and glycerin are used. Here, the sugars mean carbohydrates that are initial oxidized products of polyvalent alcohol, have one aldehyde group or ketone group, and are classified into an aldose being a sugar having the aldehyde group and a ketose being a sugar having the ketone group.

As the nitrogen sources described above, for example, ammonia gas, aqueous ammonia, ammonium salts, urea, nitrate salts, and organic nitrogen sources supplementarily used such as oilcakes, soybean hydrolysate, casein hydrolysate, other amino acids, vitamins, corn steep liquor, yeast or yeast extract, meat extract, peptide such as peptone, and various fermentation microorganisms and hydrolysates thereof are used.

As the inorganic salts described above, for example, phosphate salts, magnesium salts, calcium salts, iron salts, and manganese salts are adequately used.

As conditions, fermentation of the microorganisms can be usually carried out in ranges of a pH of 3 to 8: and a temperature of 20° C. to 65° C. The pH of the fermented liquid is adjusted in a predetermined value within the above-described range by, for example, inorganic acids or organic acids, alkaline substances, and further urea, calcium hydroxide, calcium carbonate, and ammonia gas.

As the microorganism and the cultured cells used in the continuous fermentation, eukaryotic cells or prokaryotic cells are used. Examples of the microorganism and the cultured cells include yeasts such as baker's yeast frequently used in fermentation industry, bacteria such as Escherichia coli, lactic acid bacteria, and coryneform group of bacteria, filamentous fungus, actinomyces, animal cells, and insect cells. The microorganisms and the cells used may be microorganisms and cells isolated from natural environment or may be microorganisms and cells that are partially modified by mutation or genetic modification.

The most distinctive characteristic of the eukaryotic cell is that the cell has a structure referred to as cell nucleus (nucleus) in the cell. The eukaryotic cell is clearly distinguished form prokaryotic organism that does not have the cell nucleus (nucleus). In the present invention, yeasts can be further preferably used among the eukaryotic cells. Examples of the preferable yeasts in the present invention include yeasts belonging to Genus Saccharomyces and yeasts belonging to Saccharomyces cerevisiae.

The most distinctive characteristic of the prokaryotic cell is that the cell does not have a structure referred to as cell nucleus (nucleus) in the cell. The prokaryotic cell is clearly distinguished form eukaryotic organism that has the cell nucleus (nucleus). In the present invention, lactic acid bacteria are preferably used among the prokaryotic cells.

The chemicals obtained by the apparatus for producing chemicals, that is, a substance after conversion is a substance that the microorganisms and the cultured cells produce in the fermented liquid. Examples of the chemicals may include substances that are produced in a large quantity in the fermentation industry such as alcohols, organic acids, amino acids, and nucleic acids. The apparatus for producing chemicals is also applicable for producing substances such as enzymes, antibiotics, and recombinant proteins. Examples of alcohols include ethanol, 1,3-butanediol, 1,4-butanediol, and glycerol. Examples of the organic acids may include acetic acid, lactic acid, pyruvic acid, succinic acid, malic acid, itaconic acid, and citric acid. Examples of the nucleic acids may include inosine, guanosine, and cytidine.

The substance after conversion obtained by the apparatus for producing chemicals is preferably fluid or discharged water containing at least one of a chemical product, a dairy product, a pharmaceutical product, a food product, or a brewed product. Examples of the chemical product include substances applicable for producing a chemical product by a process after the membrane filtration such as the organic acids, the amino acids, and the nucleic acids; examples of the dairy product include substances applicable for producing a dairy product by a process after the membrane filtration such as low-fat milk; examples of the pharmaceutical product include substances applicable for producing a pharmaceutical product by a process after membrane filtration such as enzymes, antibiotics, and recombinant proteins; examples of the food product include substances applicable for producing a food product by a process after the membrane filtration such as lactic acid beverages; examples of the brewed product include substances applicable for producing a brewed product by a process after the membrane filtration such as beer and shochu (Japanese distilled spirit); and example of the discharged water include discharged water after product cleaning such as dairy product cleaning discharged water and household effluent that contain a lot of organic substances.

When lactic acid is produced by the apparatus for producing chemicals 200 according to the third example, a yeast in the case of the eukaryotic cell or a lactic acid bacterium in the case of the prokaryotic cell are preferably used. Among the yeasts, yeasts introducing a gene into the cell that codes lactic acid dehydrogenase are preferable. Among the lactic acid bacteria, lactic acid bacteria that produce lactic acid in a yield to sugar of 50% or more is preferably used, and lactic acid bacteria in a yield to sugar of 80% or more is more preferably used.

Examples of the lactic acid bacteria preferably used for producing lactic acid include wild-type strains having ability of synthesizing lactic acid such as Lactobacillus, Bacillus, Pediococcus, Genus Tetragenococcus, Genus Carnobacterium, Genus Vagococcus, Genus Leuconostoc, Genus Oenococcus, Genus Atopobium, Genus Streptococcus, Genus Enterococcus, Genus Lactococcus, and Genus Sporolactobacillus.

Lactic acid bacteria that provide high yield to sugar and optical purity of lactic acid can be selectively used, and the lactic acid bacteria having ability to selectively produce D-lactic acid include D-lactic acid produce microorganisms belonging to Sporolactobacillus. As preferable specific examples, Sporolactobacillus laevolacticus or Sporolactobacillus inulinus can be used. More preferable examples include Sporolactobacillus laevolacticus ATCC 23492, ATCC 23493, ATCC 23494, ATCC 23495, ATCC 23496, ATCC 223549, IAM 12326, IAM 12327, IAM 12328, IAM 12329, IAM 12330, IAM 12331, IAM 12379, DSM 2315, DSM 6477, DSM 6510, DSM 6511, DSM 6763, DSM 6764, DSM 6771, and Sporolactobacillus inulinus JCM 6014.

Examples of lactic acid bacteria that provide high yield of L-lactic acid to sugar include Lactobacillus yamanashiensis, Lactobacillus animalis, Lactobacillus agilis, Lactobacillus aviaries, Lactobacillus easel, Lactobacillus delbruekii, Lactobacillus paracasei, Lactobacillus rhamnosu, Lactobacillus ruminis, Lactobacillus salivarius, Lactobacillus sharpeae, Pediococcus dextrinicus, and Lactococcus lactis. With selecting these lactic acid bacteria, these lactic acid bacteria can be used for producing L-lactic acid.

### (About Fermentation Conditions)

When the microorganisms and the cultured cells used for producing chemicals by the continuous fermentation require a specific nutrition for growth, the nutrition is added as a preparation or a natural product containing the nutrition. Also, a defoaming agent can be used if needed. In the production of the chemicals by the continuous fermentation, the culture liquid means liquid obtained from the result of growth of the microorganisms and the cultured cells in the fermentation feedstock. Added compositions of the fermentation feedstock may be adequately changed from the fermentation feedstock compositions at the time of starting culture so as to improve productivity of the target chemicals.

In the production of the chemicals by the continuous fermentation, when a sugar is used for the fermentation feedstock, a sugar concentration in the fermented liquid is preferably maintained in 5 g/l or less. The reason why the sugar concentration in the fermented liquid is preferably maintained in 5 g/l or less is to minimize effluence of the sugars caused by drawing the fermented liquid.

Culture of the microorganisms or the cultured cells is usually carried out in the ranges of a pH of 3 or more and 8 or less, and a temperature of 20° C. or more and 60° C. or less. The pH of the fermented liquid is adjusted in a predetermined value within the range of a pH of 3 or more and 8 or less by, for example, inorganic acids or organic acids, alkaline substances, and further urea, calcium carbonate, and ammonia gas. If a feed rate of oxygen is required to be increased, means for maintaining an oxygen concentration of 21% or more by adding oxygen to the air, pressurizing the fermented liquid, increasing stirring speed, increasing an air flow volume, and the like can be used.

In the production of the chemicals by the continuous fermentation, to carrying out cleaning, backwash, or cleaning by chemical immersion of the ceramic membrane, the ceramic membrane is required to have durability to these operations. For example, in the backwash liquid, an alkali, an acid, or an oxidizing reagent in addition to water and filtrate can be used as long as these substances do not significantly inhibit fermentation. Examples of the alkali may include an aqueous solution of sodium hydroxide and an aqueous solution of calcium hydroxide. Examples of the acid may include oxalic acid, citric acid, hydrochloric acid, and nitric acid. Examples of the oxidizing reagent may include an aqueous solution of hypochlorite salt and an aqueous solution of hydrogen peroxide. The backwash liquid can be used in high temperature if a similar temperature control to the sterilization water is carried out.

For this reason, the separation membrane module preferably has durability of a pH of 0 to 14, the alkali, the acid, or the oxidizing reagent, and further high temperature water, in addition to the durability to the steam sterilization described above.

A backwash rate of the backwash liquid is 0.5 times or more and 10 times or less and more preferably 1 time or more and 5 times or less compared with the membrane filtration rate. When the backwash rate is faster than this range, the ceramic membrane may be damaged, whereas, when slower than this range, a sufficient cleaning effect may not be obtained.

A backwash cycle of the backwash liquid can be determined by membrane pressure difference and change in membrane pressure difference. The backwash cycle is preferably 0.5 times to 12 times per hour, and more preferably 1 time to 6 times per hour. When the backwash cycle is more frequent than this range, the separation membrane may be damaged, whereas, when less frequent than this range, the sufficient cleaning effect may not be obtained.

A backwash time of the backwash liquid can be determined by the backwash cycle, the membrane pressure difference and the change in the membrane pressure difference. The backwash time is preferably 5 seconds or more and 600 seconds or less at a time, and more preferably 30 seconds or more and 300 seconds or less at a time. When the backwash time is longer than this range, the separation membrane may be damaged, whereas, when shorter than this range, the sufficient cleaning effect may not be obtained. The time required for the cleaning depends on an amount of the liquid required to be fed to the downstream of the separation membrane module, that is, a downstream volume of the separation membrane module, and thus, the downstream volume of the separation membrane module is preferably as small as possible because efficiency of the backwash is improved.

The immersion cleaning of the ceramic membrane 10 can be sequentially carried out by not discharging the backwash liquid sent to the upstream of the ceramic membrane 10 and stopping the filtration at the time of the backwash. An immersion time can be determined by an immersion cleaning cycle, the membrane pressure difference and the change in the membrane pressure difference. The immersion time is preferably one minute or more and 24 hours or less at one time, and more preferably in a range of 10 minutes to 12 hours at one time.

In the production of the chemicals by the continuous fermentation, after batch culture or fed-batch culture is carried out to increase a microorganism concentration, the continuous fermentation (drawing) may be started. Alternatively, after the microorganism concentration is increased, high concentration microorganism body may be seeded and continuous fermentation may be carried out with start of culture. In the production of the chemicals by the continuous fermentation, feed of the feedstock culture liquid and draw of cultured substance can be carried out from adequate timing. Start timing of the feed of the feedstock culture liquid and the draw of cultured substance is not necessarily the same. The feed of the feedstock culture liquid and the draw of cultured substance may be continuous or intermittent.

The microorganism growth may be continuously carried out by adding the nutrition required for microorganism growth to the feedstock culture liquid. It is a preferable aspect for obtaining efficient productivity that a concentration of the microorganisms and the cultured cells in the fermented liquid is maintained in a high state in a range as long as a rate of extinction caused by inadequate environment of the fermented liquid for the growth of the microorganisms and the cultured cells does not become high. As one example, the concentration of the microorganisms and the cultured cells in the fermented liquid to ferment D-lactic acid by using Sporolactobacillus laevolacticus JCM2513 (SL strain) being one of the lactic acid bacteria is 5 g/L or more as a dry weight. Excellent productivity can be obtained by maintaining this concentration.

In the production of the chemicals by the continuous fermentation, the microorganisms and the cultured cells can be drawn from the fermentation tank if needed. For example, when the concentration of the microorganisms and the cultured cells in the fermentation tank becomes too high, clogging of the ceramic membrane is easy to occur. The clogging of the separation membrane can be avoided by drawing the microorganisms and the cultured cells. Production performance of the chemicals may vary depending on the concentration of the microorganisms and the cultured cells in the fermentation tank. However, the production performance can be maintained by drawing the microorganisms and the cultured cells using the production performance as an index.

In the production of the chemicals by the continuous fermentation, the number of the fermentation reaction tanks does not matter in the continuous culture operation carried out with growing fresh microorganisms having ability of fermentation production as long as the operation is a continuous culture method for producing products with growing microorganisms. In the production of the chemicals by the continuous fermentation, usually, the continuous culture operation is preferably carried out in a single fermentation reaction tank because of culture management. However, a plurality of fermentation reaction tanks can be used for the reason that a volume of the fermentation reaction tank is small or the like. In this case, high productivity of fermentation products can be obtained when the continuous culture is carried out by parallelly or serially connecting the fermentation reaction tanks with piping.

### EXAMPLES

### First Reference Example

### Preparation of Monolith Membrane

Extrusion molding product that includes alumina as a main component and is a cylindrical product having an outer diameter of 36 mm and a length of 200 mm and including 37 circular through-bores having a diameter of 3 mm in a longitudinal direction of the substrate was produced and the product was sintered at 1250° C. for 1 hour to obtain a monolith substrate.

Subsequently, a slurry to form a first separation function layer was prepared in a manner that 10% by mass of a frit containing SiO₂/Al₂O₃ as the main component and 10% by mass or less of ZrO₂ and having an average particle diameter of 1 μm or less ground by a boll mill or other equipment was mixed with 90% by mass of alumina having an average particle diameter of 1.2 μm, and 0.5% by mass of ammonium polycarboxylate and 0.5% by mass of a polysaccharide binder as organic binders and 80% by mass of water to the total mass were added to the mixture. The slurry was circulated in the through-bores of the monolith substrate. When a membrane forming material having an amount that forms a membrane having a thickness of 150 μm was attached to the through-bores of the monolith substrate, the circulation was stopped. Thereafter, the slurry was discharged from the monolith substrate and the membrane forming material was dried under vacuum for 10 minutes. After the membrane forming material was further dried at 60° C. for 20 hours and sintered at 960° C. for 1 hour to form a first separation function layer on the monolith substrate.

Further, a slurry to form a second separation membrane function layer was prepared so that each of 0.5% by mass of ammonium polycarboxylate and a 1.0% by mass of polycarboxylic acid binding agent, and 95% by mass of water was added to 3.5% by mass of alumina having an average particle diameter of 0.6 μm. By using this membrane formation slurry, the film formation was carried out by the method described above until the membrane forming material having an amount that forms a membrane having a thickness of 30 μm was attached. After the membrane forming material was dried at 60° C. for 20 hours, the membrane forming material was sintered at 1400° C. for 1 hour to stack the second separation function layer, and thus the monolith membrane was obtained.

### Second Reference Example

### Preparation of Separation Membrane Module

The monolith membrane obtained in the first reference example was placed in a module container made of stainless steel having an inner diameter of 40 mm, and an EPDM O-ring was located between the module container and the separation membrane to prepare the separation membrane module 1.

### Third Reference Example

### Continuous Fermentation of D-Lactic Acid

The continuous fermentation of D-lactic acid was carried out in accordance with the following conditions. The continuous fermentation was carried out using the apparatus for producing chemicals 200 in FIG. 10.

Fermentation tank capacity: 2 (L)
Fermentation tank effective capacity: 1.5 (L)
Temperature adjustment: 37 (° C.).
Fermentation tank air flow volume: Nitrogen 0.2 (L/min)
Fermentation tank stirring rate: 600 (rpm)
pH adjustment: Adjusted to pH 6 by 3N Ca(OH)₂
Lactic acid fermentation culture medium feed: Added by controlling so that a liquid amount in the fermentation tank is constant at about 1.5 L
Liquid amount circulated by fermented liquid circulation device: 2 (L/min)
Membrane filtration flow rate control: Flow volume control by suction pump
Intermittent filtration process: Periodic operation of filtration process (9 minutes)-filtration stopping process (1 minutes)
Membrane filtration flux: Variable so that transmembrane pressure difference of 500 kPa or less in range of 0.01 (m/day) or more and 5 (m/day) or less. When the transmembrane pressure difference continuously increased beyond the range, the continuous fermentation was stopped.

Culture medium compositions were as shown in Table 1.

TABLE 1
Culture medium for fermentation of lactic acid

| Composition | Content | |
|---|---|---|
| Glucose | 100 | g |
| Yeast Nitrogen base W/O amino acid (Difco) | 6.7 | g |
| 19 standard amino acids except leucine | 152 | mg |
| Leucine | 760 | mg |
| Inositol | 152 | mg |
| p-aminobenzoic acid | 16 | mg |
| Adenyl | 40 | mg |
| Uracil | 152 | mg |
| Water | 892 | g |

Sporolactobacillus laevolacticus JCM2513 (SL strain) was used as the microorganism. Evaluation of the concentration of lactic acid being the product was carried out using HPLC described below in accordance with the following conditions.

Column: Shim-Pack SPR-H (manufactured by Shimadzu Corporation)
Mobile phase: 5 mM p-toluenesulfonic acid (0.8 mL/min)
Reaction phase: 5 mM p-toluenesulfonic acid, 20 mM bis-tris, and 0.1 mM EDTA.2Na (0.8 mL/min)
Detection method: Electric conductivity
Column temperature: 45° C.
Analysis of an optical purity of the lactic acid was carried out by the following conditions.
Column: TSK-gel Enantio L1 (manufactured by Tosoh Corporation)
Mobile phase: 1 mM aqueous copper sulfate solution
Flux: 1.0 mL/minute
Detection method: UV 254 nm

### Temperature: 30° C.

The optical purity of L-lactic acid was calculated by Formula (I).

Optical purity (%)=100×(L−D)/(D+L) (i)

The optical purity of D-lactic acid was calculated by Formula (II).

Optical purity (%)=100×(D−L)/(D+L) (ii)

Here, L represents a concentration of L-lactic acid and D represents a concentration of D-lactic acid.

As the culture, first, shaking culture of the SL strain was carried out in 5 ml of lactic acid fermentation culture medium in a test tube for one night (pre-culture before pre-culture before pre-culture). The obtained culture liquid was inoculated to 100 ml of fresh lactic acid fermentation culture medium, and shaking culture of the inoculated medium was carried out in 500 ml of Sakaguchi flask at 30° C. for 24 hours (pre-culture before pre-culture). A liquid of the pre-culture before pre-culture was inoculated after the culture medium was poured into the fermentation tank 100 of the apparatus for producing chemicals 200 illustrated in FIG. 10. The mixture in the fermentation tank 100 was stirred by the attached stirring device 103, and air flow volume adjustment, temperature adjustment, and pH adjustment in the fermentation tank 100 were carried out. The culture was carried out for 24 hours without operating the circulation pump 101 (pre-culture). Just after the completion of the pre-culture, the production of D-lactic acid by the continuous fermentation was carried out by operating the circulation pump 101, continuously feeding the lactic acid fermentation culture medium in addition to the operating condition at the time of the pre-culture, and carrying out the continuous culture with controlling permeation rate so that the amount of the fermented liquid in the continuous fermentation device is 1.5 L. The permeation rate at the time of the continuous fermentation test was controlled by the filtration pump 109 so that a filtration amount is the same as the flow rate of the fed fermentation culture medium. Adequately, a concentration of produced D-lactic acid and a concentration of remaining glucose in the membrane permeated fermented liquid were measured.

### First Comparative Example

The monolith membrane obtained in the first reference example is placed in an electric oven to heat to 300.0° C. The heating rate was 15.0° C. per minute. The temperature was maintained for 30 minutes after reaching 300.0° C., and thereafter, the power source of the electric oven was turned off. The inside of the oven was cooled by a ventilation fan attached to the electric oven. The internal temperature became 25.0° C. for 20 minutes after the power source of the electric oven was turned off. An average rate of temperature change at the time of cooling was 13.8° C. per minute. The monolith membrane for which the hot-air sterilization was carried out in this procedure generated cracks because the monolith membrane was not able to endure the rapid temperature change.

By using this monolith membrane, a separation membrane module was produced by a similar procedure to the second reference example. Thereafter, sterilization water was filled in the downstream of the separation membrane module; sterilization air was sent to the upstream of the separation membrane module; and the upstream of the separation membrane module was pressurized so that a gauge pressure is 50 kPa; and then the pressure was maintained for one minute. At this time, whether bubbles from the downstream of the separation membrane module were generated or not was ascertained. Hereinafter, this procedure is referred to as an air leak test. A module in which bubbles were generated in the downstream of the separation membrane within one minute was impaired in air tightness of the separation membrane (leaked), and thus, the module was determined to fail the air leak test.

The air leak test of this separation membrane module was carried out and leakage caused by the cracks in the monolith membrane was ascertained. The sterilization process conditions at this time were collectively listed in Table 2.

### Second Comparative Example

A separation membrane module was prepared from the monolith membrane described in the first reference example by the method described in the second reference example, and connected to the sterilization device 2A illustrated in FIG. 6. A temperature T1 of the upstream of the separation membrane module 1 was 25.0° C. In the sterilization water control part 3, a temperature Tw of the sterilization water was set to 30.0° C. and the state was a liquid phase state, and continuous feed of the sterilization water was started to the upstream of the lower part of the separation membrane module 1. |T1−Tw| was equal to 5.0° C. Thereafter, with controlling so that ΔT1 is 7.0° C. per minute, the sterilization water was continuously fed and an internal temperature of the separation membrane module was raised. After T1 reached to 100° C., the sterilization water was controlled so as to be pressurized. After T1 reached to 121° C., the state of 121° C. was maintained for 20 minutes. Thereafter, the separation membrane module 1 was cooled by continuously feeding the sterilization water in the state of pressurization with controlling so that ΔT1 is 7.0° C. per minute to the upstream of the separation membrane module 1. The control was carried out so that the pressure of the sterilization water backed to normal when T1 reached to 100° C. or less. Feed of the sterilization water was stopped when T1 reached to 37° C.

Thereafter, the air leak test of the separation membrane module 1 was carried out by a similar procedure to a first comparative example, and leakage caused by the cracks in the monolith membrane was ascertained. The sterilization process conditions at this time were collectively listed in Table 2.

### Third Comparative Example

Similar to the second comparative example, a separation membrane module 1 using the monolith membrane was prepared and the module was connected to the sterilization device 2 illustrated in FIG. 7. A temperature T1 of the upstream of the separation membrane module 1 was 50.0° C. In the sterilization water control part 3, a temperature Tw of the sterilization water was set to 90.0° C. and the state was a gas phase state, and continuous feed of the sterilization water was started to the upstream of the upper part of the separation membrane module 1. |T1−Tw| was equal to 40.0° C. Thereafter, with controlling so that ΔT1 is 5.5° C. per minute, the sterilization water was continuously fed and an internal temperature of the separation membrane module was raised. After T1 reached to 100° C., the sterilization water was controlled so as to be pressurized. After T1 reached to 121° C., the state of 121° C. was maintained for 20 minutes. Thereafter, the separation membrane module 1 was cooled by continuously feeding the sterilization water in the state of pressurization with controlling so that ΔT1 is 5.5° C. per minute to the upstream of the separation membrane module 1. The control was carried out so that the pressure of the sterilization water backed to normal when T1 reached to 100° C. or less. Feed of the sterilization water was stopped when T1 reached to 37° C.

Thereafter, the air leak test of the separation membrane module 1 was carried out by a similar procedure to the first comparative example, and leakage caused by the cracks in the monolith membrane was ascertained. The sterilization process conditions at this time were collectively listed in Table 2.

### First Example

Similar to the second comparative example, a separation membrane module 1 using the monolith membrane was prepared and the module was connected to the sterilization device 2B illustrated in FIG. 7 to carry out the sterilization process. The temperature T1 of the upstream of the separation membrane module 1 was 20.0° C. In the sterilization water control part 3, a temperature Tw of the sterilization water was set to 30.0° C. and the state was a liquid phase state, and continuous feed of the sterilization water to the upstream of the lower part of the separation membrane module 1 was started. |T1−Tw| was equal to 10.0° C.

Thereafter, with controlling so that ΔT1 is 5.5° C. per minute, the sterilization water was continuously fed and the internal temperature of the separation membrane module 1 was raised. After T1 reached to 100° C., the sterilization water was controlled so as to pressurize the sterilization water. After T1 reached to 121° C., the state of 121° C. was maintained for 20 minutes. Thereafter, the separation membrane module 1 was cooled by continuously feeding the sterilization water in the state of pressurization with controlling so that ΔT1 is 5.5° C. per minute to the upstream of the separation membrane module 1. The control was carried out so that the pressure of the sterilization water backed to normal when T1 reached to 100° C. or less. Feed of the sterilization water was stopped when T1 reached to 37° C.

Thereafter, continuous fermentation of D-lactic acid was carried out in accordance with the description in the third reference example. When the continuous fermentation was carried out in these conditions, it was ascertained that the continuous fermentation for 400 hours was possible from the start of the continuous fermentation. The results of the continuous fermentation are collectively listed in Table 2.

To ascertain the durability to the steam sterilization of the separation membrane module, the sterilization process of the separation membrane module was repeatedly carried out by a method similar to the method described above. As a result, in the method described in this Example, no crack was generated in the monolith membrane and 10 times of repeated sterilization processes were possible.

### Second Example

Similar to the first example, a separation membrane module 1 using the monolith membrane was prepared and the module was connected to the separation sterilization device 2 illustrated in FIG. 1 to carry out the sterilization process. In the sterilization process, sterilization process of the separation membrane module was carried out by controlling the upstream of the separation membrane module so that an initial temperature T and a rate of temperature change ΔT were similar to the first example and feeding the sterilization water in a gas phase state from the upper part of the upstream of the separation membrane module 1. At 100° C. or more, the sterilization water in the gas phase state was used with pressurization. Sterilization water in a liquid phase state formed by condensation was permitted to be mixed in the sterilization water in the gas phase state. Hereinafter, similarly, the sterilization water in the liquid phase state may be mixed in the sterilization water in the gas phase state when the sterilization water in the gas phase state is used in other Examples.

Thereafter, the continuous fermentation of D-lactic acid was carried out in accordance with the description of the third reference example. When the continuous fermentation was carried out in these conditions, it was ascertained that the continuous fermentation for 400 hours was possible from the start of the continuous fermentation. The results of the continuous fermentation are collectively listed in Table 2.

Similar to the first example, durability to the steam sterilization of the separation membrane module was ascertained. As a result, in the method described in this Example, no crack was generated in the monolith membrane and 10 times of repeated sterilization processes were possible.

### Third Example

Similar to the first example, a separation membrane module 1 using the monolith membrane was prepared and the sterilization device 2C being a device illustrated in FIG. 10 was connected to the lower part of the separation membrane module 1 to carry out the sterilization process. In the separation membrane module 1, the temperature T1 of the upstream was 20.2° C. and the temperature T2 of the downstream of was 20.4° C. In the sterilization water control part 3, a temperature Tw of the sterilization water was set to 30.0° C. and the state was a liquid phase state, and continuous feed of the sterilization water was started to the upstream and the downstream of the lower part of the separation membrane module 1. |T1−Tw| was equal to 9.6° C. and |T2−Tw| was equal to 9.4° C.

Thereafter, with controlling so that each of ΔT1 and ΔT2 were 5.5° C. per minute, the sterilization water was continuously fed and the internal temperature of the separation membrane module 1 was raised. After either one of T1 or T2 reached to 100° C., the sterilization water was controlled so as to be pressurized. After T1 and T2 reached to 121° C., a state in which both T1 and T2 were 121° C. or more was maintained for 20 minutes. Thereafter, the separation membrane module 1 was cooled by continuously feeding the sterilization water in the state of pressurization with controlling so that Δ1 and ΔT2 were 5.5° C. per minute to the upstream and the downstream of the separation membrane module 1. The control was carried out so that the pressure of the sterilization water backed to normal when T1 and T2 reached to 100° C. or less. Feed of the sterilization water was stopped when T1 and T2 reached to 37° C.

Thereafter, the continuous fermentation of D-lactic acid was carried out in accordance with the description of the third reference example. When the continuous fermentation was carried out in these conditions, it was ascertained that the continuous fermentation for 400 hours was possible from the start of the continuous fermentation. The results of the continuous fermentation are collectively listed in Table 2.

Similar to the first example, durability to the steam sterilization of the separation membrane module was ascertained. As a result, in the method described in this Example, no crack was generated in the monolith membrane and 10 times of repeated sterilization processes were possible.

### Fourth Example

Similar to the first example, a separation membrane module 1 using the monolith membrane was prepared and the module was connected to the sterilization device 2C illustrated in FIG. 10. A sterilization process of the separation membrane module was carried out by feeding the sterilization water in a gas phase state having a controlled temperature and pressure to the upstream and the downstream of the upper part of the separation membrane module 1 so that an initial temperature T and a rate of temperature change ΔT were similar to the third example.

Thereafter, the continuous fermentation of D-lactic acid was carried out in accordance with the description of the third reference example. When the continuous fermentation was carried out in these conditions, it was ascertained that the continuous fermentation for 400 hours was possible from the start of the continuous fermentation. The results of the continuous fermentation are collectively listed in Table 2.

Similar to the first example, durability to the steam sterilization of the separation membrane module was ascertained. As a result, in the method described in this Example, no crack was generated in the monolith membrane and 10 times of repeated sterilization processes were possible.

### Fifth Example

Similar to the first example, a separation membrane module 1 using the monolith membrane was prepared and the sterilization device 2D illustrated in FIG. 11 was connected to the lower part of the separation membrane module 1. The temperature T1 of the upstream of the separation membrane module 1 was 20.0° C. In the sterilization water control part 3, a temperature Tw of the sterilization water was set to 30.0° C. and the state was a liquid phase state, and continuous feed of the sterilization water was started to the upstream of the lower part of the separation membrane module 1. |T1−Tw| was equal to 10.0° C. Subsequently, the sterilization water in the liquid phase state controlled so that a rate of temperature change ΔT was similar to the first example was continuously fed to the upstream of the separation membrane module 1. In addition, the sterilization process of the separation membrane module 1 was carried out by applying a pressure of 100 kPa to the upstream of the separation membrane module 1 by the sterilization water feed pump 8 and permeating the monolith membrane from the upstream to the downstream with the sterilization water.

Thereafter, the continuous fermentation of D-lactic acid was carried out in accordance with the description of the third reference example. When the continuous fermentation was carried out in these conditions, it was ascertained that the continuous fermentation for 400 hours was possible from the start of the continuous fermentation. The results of the continuous fermentation are collectively listed in Table 2.

Similar to the first example, durability to the steam sterilization of the separation membrane module was ascertained. As a result, in the method described in this Example, no crack was generated in the monolith membrane and 10 times of repeated sterilization processes were possible.

### Sixth Example

Similar to the first example, a separation membrane module 1 using the monolith membrane was prepared and the sterilization device 2D illustrated in FIG. 11 was connected to the lower part of the separation membrane module 1. The temperature T1 of the upstream of the separation membrane module was 20.0° C. In the sterilization water control part 3, a temperature Tw of the sterilization water was set to 30.0° C. and the state was a liquid phase state, and continuous feed of the sterilization water was started to the upstream of the lower part of the separation membrane module 1. |T1−Tw| was equal to 10.0° C. Subsequently, the sterilization water in the liquid phase state controlled so that a rate of temperature change ΔT1 was 3.5° C. per minute was continuously fed to the upstream of the separation membrane module 1. In addition, the sterilization process of the separation membrane module 1 was carried out by applying a pressure of 100 kPa to the upstream of the separation membrane module 1 by the sterilization water feed pump 8 and permeating the monolith membrane from the upstream to the downstream with the sterilization water.

Thereafter, the continuous fermentation of D-lactic acid was carried out in accordance with the description of the third reference example. When the continuous fermentation was carried out in these conditions, it was ascertained that the continuous fermentation for 400 hours was possible from the start of the continuous fermentation. The results of the continuous fermentation are collectively listed in Table 2.

Similar to the first example, durability to the steam sterilization of the separation membrane module was ascertained. As a result, in the method described in this Example, 10 times of repeated sterilization processes were possible.

### Seventh Example

Similar to the first example, a separation membrane module 1 using the monolith membrane was prepared and the sterilization device 2D illustrated in FIG. 11 was connected to the lower part of the separation membrane module 1. The temperature T1 of the upstream of the separation membrane module was 20.0° C. In the sterilization water control part 3, a temperature Tw of the sterilization water was set to 45.0° C. and the state was a liquid phase state, and continuous feed of the sterilization water was started to the upstream of the lower part of the separation membrane module 1. |T1−Tw| was equal to 25.0° C. Subsequently, the sterilization water in the liquid phase state controlled in a temperature so that a rate of temperature change ΔT was similar to the first example was continuously fed to the upstream of the separation membrane module 1. In addition, the sterilization process of the separation membrane module 1 was carried out by applying a pressure of 100 kPa to the upstream of the separation membrane module 1 by the sterilization water feed pump 8 and permeating the monolith membrane from the upstream to the downstream with the sterilization water.

Thereafter, the continuous fermentation of D-lactic acid was carried out in accordance with the description of the third reference example. When the continuous fermentation was carried out in these conditions, it was ascertained that the continuous fermentation for 400 hours was possible from the start of the continuous fermentation. The results of the continuous fermentation are collectively listed in Table 2.

Similar to the first example, durability to the steam sterilization of the separation membrane module was ascertained. As a result, in the method described in this Example, 10 times of repeated sterilization processes were possible.

### Eighth Example

Similar to the first example, a separation membrane module 1 using the monolith membrane was prepared and the sterilization device 2D illustrated in FIG. 11 was connected to the lower part of the separation membrane module 1. The temperature T1 of the upstream of the separation membrane module 1 was 20.0° C. In the sterilization water control part 3, a temperature Tw of sterilization water was set to 25.0° C. and the state was a liquid phase state, and continuous feed of the sterilization water was started to the upstream of the lower part of the separation membrane module 1. |T1−Tw| was equal to 5.0° C. Subsequently, the sterilization water in the liquid phase state controlled in a temperature so that a rate of temperature change ΔT were similar to the first example was continuously fed to the upstream of the separation membrane module 1. In addition, the sterilization process of the separation membrane module 1 was carried out by applying a pressure of 100 kPa to the upstream of the separation membrane module 1 by the sterilization water feed pump 8 and permeating the monolith membrane from the upstream to the downstream with the sterilization water.

Thereafter, the continuous fermentation of D-lactic acid was carried out in accordance with the description of the third reference example. When the continuous fermentation was carried out in these conditions, it was ascertained that the continuous fermentation for 400 hours was possible from the start of the continuous fermentation. The results of the continuous fermentation are collectively listed in Table 2.

Similar to the first example, durability to the steam sterilization of the separation membrane module was ascertained. As a result, in the method described in this Example, 10 times of repeated sterilization processes were possible.

### Ninth Example

Similar to the first example, a separation membrane module 1 using the monolith membrane was prepared and the sterilization device 2C illustrated in FIG. 10 was connected to the lower part of the separation membrane module 1. In the separation membrane module 1, both of the temperature T1 of the upstream and the temperature T2 of the downstream were 20.0° C. In the sterilization water control part 3, a temperature Tw of the sterilization water was set to 90.0° C. and the state was a gas phase state, and continuous feed of the sterilization water was started to the upstream and the downstream of the upper part of the separation membrane module. |T1−Tw| and |T2−Tw| were equal to 10.0° C. Thereafter, with controlling so that ΔT1 and ΔT2 were raised at 5.5° C. per minute, the sterilization water was continuously fed and the internal temperature of the separation membrane module 1 was raised. After either one of T1 or T2 reached to 100° C., the sterilization water was controlled so as to be pressurized. After T1 and 12 reached to 200.0° C., the feed of the sterilization water to the downstream of the separation membrane module 1 was stopped and the temperature was maintained for 5 minutes by permeating from the upstream to the downstream with the sterilization water. Thereafter, the separation membrane module 1 was cooled by restarting the feed of the sterilization water to the downstream of the separation membrane module and continuously feeding the sterilization water in the state of pressurization with controlling so that ΔT1 and ΔT2 were 5.5° C. per minute to the upstream and the downstream of the separation membrane module. The control was carried out so that the pressure of the sterilization water backed to normal when T1 and T2 reached to 100° C. or less. Feed of the sterilization water was stopped when T1 and T2 reached to 37° C.

Subsequently, sterilization process of the separation membrane module 1 was carried out by feeding the sterilization water in a gas phase state having a controlled temperature and pressure similar to the second example to the upstream of the separation membrane module 1 and permeating the monolith membrane from the upstream to the downstream with the sterilization water. The sterilization water was discharged from the downstream of the separation membrane module 1 and was returned to the sterilization water control part 3 through a sterilization filter.

Thereafter, the continuous fermentation of D-lactic acid was carried out in accordance with the description of the third reference example. When the continuous fermentation was carried out in these conditions, it was ascertained that the continuous fermentation for 400 hours was possible from the start of the continuous fermentation. The results of the continuous fermentation are collectively listed in Table 2.

Similar to the first example, durability to the steam sterilization of the separation membrane module was ascertained. As a result, in the method described in this Example, 10 times of repeated sterilization processes were possible.

TABLE 2

| | | | | | | | | | | First | Second | Third |
|---|---|---|---|---|---|---|---|---|---|---|---|---|
| | First | Second | Third | Fourth | Fifth | Sixth | Seventh | Eighth | Ninth | Comp. | Comp. | Comp. |
| | Exam- | Exam- | Exam- | Exam- | Exam- | Exam- | Exam- | Exam- | Exam- | Exam- | Exam- | Exam- |
| | ple | ple | ple | ple | ple | ple | ple | ple | ple | ple | ple | ple |
| Supply of | Upstream only | | Upstream and | | Sterilization water permeated from upstream to | | | | | (Electric | Upstream | Upstream |
| sterilization | | | downstream | | downstream | | | | | oven) | only | only |
| water | | | | | | | | | | | | |
| Phase of | Liquid | Gas | Liquid | Gas | Liquid | Liquid | Liquid | Liquid | Gas | | Liquid | Liquid |
| sterilization | phase | phase | phase | phase | phase | phase | phase | phase | phase | | phase | phase |
| water | | | | | | | | | | | | |
| \|T-Tw\| [° C.] | 10.0 | 10.0 | 9.6 | 9.6 | 10.0 | 10.0 | 25.0 | 10.0 | 10.0 | (Unmea- | 5.0 | 40.0 |
| (Max. value | | | | | | | | | | sured) | | |
| for multiple | | | | | | | | | | | | |
| measurements) | | | | | | | | | | | | |
| Rate of T | 5.5 | 5.5 | 5.5 | 5.5 | 5.5 | 3.5 | 5.5 | 5.5 | 5.5 | 15.0 | 7.0 | 5.5 |
| change | | | | | | | | | | | | |
| [° C./min] | | | | | | | | | | | | |
| (Max. value | | | | | | | | | | | | |
| for multiple | | | | | | | | | | | | |
| measurements) | | | | | | | | | | | | |
| Fermentation | 400 | 400 | 400 | 400 | 400 | 400 | 400 | 400 | 400 | (Impos- | (Impos- | (Impos- |
| time [hr] | | | | | | | | | | sible) | sible) | sible) |
| Total | 4800 | 4800 | 4800 | 4800 | 4800 | 4800 | 4800 | 4800 | 4800 | — | — | — |
| amount of | | | | | | | | | | | | |
| glucose | | | | | | | | | | | | |
| provided [g] | | | | | | | | | | | | |
| Total | 4160 | 4210 | 4090 | 4270 | 4330 | 4260 | 4300 | 4410 | 4370 | — | — | — |
| amount of | | | | | | | | | | | | |
| produced D- | | | | | | | | | | | | |
| lactic acid [g] | | | | | | | | | | | | |
| Remaining | 70 | 90 | 100 | 70 | 60 | 70 | 80 | 60 | 70 | — | — | — |
| glucose [g] | | | | | | | | | | | | |
| Production | 6.9 | 7.0 | 6.8 | 7.1 | 7.2 | 7.1 | 7.2 | 7.4 | 7.3 | | | |
| rate of D- | | | | | | | | | | | | |
| lactic acid | | | | | | | | | | | | |
| [g/L/hr] | | | | | | | | | | | | |

### INDUSTRIAL APPLICABILITY

When the method of sterilizing the separation membrane module is used, the sterilization process can be carried out while reducing the damage of the separation membrane module including the separation membrane containing at least ceramic, and can be suitably used in a purification process of food products and pharmaceutical products and the like. When the sterilization process device which achieves the method of sterilizing or the apparatus for producing chemicals which is an application example thereof are used high productivity can be stably maintained for long periods and the continuous fermentation in which the sterilization is processable can be carried out, and thus, chemicals being fermentation products can be stably produced in low cost in the wide range of fermentation industry under simple operation conditions.

1.-9. (canceled)

10. A method of sterilizing a separation membrane module comprising a separation membrane containing at least ceramic, by using sterilization water, the method comprising:
a temperature raising step of raising a temperature of the separation membrane module to a predetermined sterilization temperature by feeding the sterilization water to the separation membrane module and controlling temperature and pressure of the fed sterilization water so that the temperature of the separation membrane module rises at 6.0° C. or less per minute; and
a sterilization step of sterilizing the separation membrane module at a predetermined temperature for a predetermined time after the temperature of the separation membrane module reaches the predetermined sterilization temperature.

11. The method according to claim 10, further comprising:
a temperature measurement step of measuring a temperature T of the separation membrane module; and
an initial temperature control step of controlling T and/or a temperature Tw of the sterilization water so that Tw satisfies |T−Tw|≦30.0° C.,
wherein the temperature raising step is carried out after the initial temperature control step.

12. The method according to claim 10;
wherein the temperature measurement step measures temperature of an upstream of the separation membrane module which is a fed side of raw liquid being a process target as the temperature T; and
the temperature raising step and the sterilization step feed the sterilization water to the upstream of the separation membrane module.

13. The method according to claim 10;
wherein the temperature measurement step measures any one of temperatures T of an upstream and a downstream of the separation membrane module as the temperature T; and
the temperature raising step and the sterilization step feed the sterilization water to the upstream and downstream of the separation membrane module.

14. A method of sterilizing a separation membrane module comprising a separation membrane containing at least ceramic, by using sterilization water, the method comprising:
a temperature raising step of raising a temperature of the separation membrane module to a predetermined sterilization temperature by feeding the sterilization water to an upstream where raw liquid being a process target of the separation membrane module is fed and to a downstream where filtrate after processing is collected and controlling temperature and pressure of the fed sterilization water so that the temperature of the upstream and the downstream of the separation membrane module rises at 6.0° C. or less per minute; and
a sterilization step of sterilizing the separation membrane module at a predetermined temperature for a predetermined time after the temperature of the upstream and the downstream of the separation membrane module reaches the predetermined sterilization temperature.

15. The method according to claim 14, further comprising:
a temperature measurement step of measuring a temperature T1 of the upstream and a temperature T2 of downstream of the separation membrane module; and
an initial temperature control step of controlling the T1 and/or the T2 and/or a temperature Tw1 of the sterilization water fed to the upstream of the separation membrane module and temperature Tw2 of the sterilization water fed to the downstream of the separation membrane module so that the Tw1 and Tw2 satisfy |T1−Tw1|≦30.0° C. and |T2−Tw2|≦30.0° C.,
wherein the temperature raising step is carried out after the initial temperature control step.

16. The method according to claim 10, further comprising a cooling step of cooling the separation membrane module after the sterilization step so that temperature of the separation membrane module drops at 6.0° C. or less per minute.

17. A sterilization device for sterilizing a separation membrane module comprising a separation membrane comprising at least ceramic, the device comprising:
a temperature measurement device that measures temperature of the separation membrane module; and
a sterilization water control part that generates liquid phase or gas phase sterilization water having a controlled temperature and pressure and to feed the sterilization water to the separation membrane module,
wherein the sterilization water control part feeds the sterilization water so that temperature of the separation membrane module rises or drops at 6.0° C. or less per minute.

18. An apparatus for producing chemicals comprising:
a separation membrane module comprising a separation membrane comprising at least ceramic;
a sterilization device that sterilizes a separation membrane module comprising a separation membrane comprising at least ceramic, the sterilization device comprising: a temperature measurement device that measures temperature of the separation membrane module; and a sterilization water control part that generates liquid phase or gas phase sterilization water having a controlled temperature and pressure and to feed the sterilization water to the separation membrane module, wherein the sterilization water control part feeds the sterilization water so that temperature of the separation membrane module rises or drops at 6.0° C. or less per minute;
a fermentation tank that converts a feedstock into fermented liquid containing chemicals by fermentation culture of the feedstock by microorganisms; and
a fermented liquid circulation means that sends the fermented liquid from the fermentation tank to the separation membrane module.

19. The method according to claim 14, further comprising a cooling step of cooling the separation membrane module after the sterilization step so that temperature of the separation membrane module drops at 6.0° C. or less per minute.

20. The method according to claim 11;
wherein the temperature measurement step measures temperature of an upstream of the separation membrane module which is a fed side of raw liquid being a process target as the temperature T; and
the temperature raising step and the sterilization step feed the sterilization water to the upstream of the separation membrane module.

21. The method according to claim 11;
wherein the temperature measurement step measures any one of temperatures T of an upstream and a downstream of the separation membrane module as the temperature T; and
the temperature raising step and the sterilization step feed the sterilization water to the upstream and downstream of the separation membrane module.

22. The method according to claim 11, further comprising a cooling step of cooling the separation membrane module after the sterilization step so that temperature of the separation membrane module drops at 6.0° C. or less per minute.

23. The method according to claim 12, further comprising a cooling step of cooling the separation membrane module after the sterilization step so that temperature of the separation membrane module drops at 6.0° C. or less per minute.

24. The method according to claim 13, further comprising a cooling step of cooling the separation membrane module after the sterilization step so that temperature of the separation membrane module drops at 6.0° C. or less per minute.

25. The method according to claim 14, further comprising a cooling step of cooling the separation membrane module after the sterilization step so that temperature of the separation membrane module drops at 6.0° C. or less per minute.

26. The method according to claim 15, further comprising a cooling step of cooling the separation membrane module after the sterilization step so that temperature of the separation membrane module drops at 6.0° C. or less per minute.
